(19) United States
(12) Patent Application Publication
(10) Pub. No.: US 2024/0124840 A1
(43) Pub. Date: Apr. 18, 2024

(54) GENETICALLY MODIFIED CELLS AND USES THEREOF FOR PREVENTION OF ACUTE GRAFT-VERSUS-HOST DISEASE

(71) Applicant: Ohio State Innovation Foundation, Columbus, OH (US)

(72) Inventors: Parvathi RANGANATHAN, Columbus, OH (US); Ramiro GARZON, Columbus, OH (US)

(21) Appl. No.: 18/278,317

(22) PCT Filed: Feb. 22, 2022
(86) PCT No.: PCT/US2022/017277

§ 371 Date: Aug. 22, 2023

Related U.S. Application Data
(60) Provisional application No. 63/151,860, filed on Feb. 22, 2021.

(51) Int. Cl. C12N 5/0783; C12N 9/22; C12N 15/113

(52) U.S. Cl. CPC C12N 5/0636; C12N 9/22; C12N 15/113; C12N 2310/113; C12N 2310/20; C12N 2510/00

(57) ABSTRACT

The present disclosure relates to compositions and methods for preventing and treating acute graft-versus-host disease.

### CROSS-REFERENCE TO RELATED APPLICATIONS

This application claims the benefit of U.S. Provisional Application No. 63/151,860, filed Feb. 22, 2021, which is expressly incorporated herein by reference in its entirety.

### FIELD

The present disclosure relates to genetically modified cells and uses thereof.

### BACKGROUND

More than 8000 patients receive an allo-hematopoietic stem cell transplant (HSCT) annually in the US alone as a cure for hematologic malignancies and other primary bone marrow disorders. However, the major barrier for the success of allo-HCT is the high incidence of acute graft-versus-host disease (aGVHD) and its associated morbidity and mortality. The pathogenesis of aGVHD involves recognition of host antigens by donor T cells followed by expansion, migration, and finally end-organ damage due to the combination of inflammatory cytokine secretion and direct cytotoxic effects. Acute GVHD is clinically characterized by damage to the skin, liver, and gastrointestinal (GI) tract. The incidence and severity of aGVHD depends on the degree of histocompatibility between donor and recipient, the donor graft source (peripheral blood or bone marrow), recipient's age, and GVHD prophylactic regimen.

What is needed are new compositions and methods for preventing and treating aGVHD.

### SUMMARY

Disclosed herein are compositions, cells, and methods for the treatment and/or prevention of acute graft-versus-host disease.

In some aspects, disclosed herein is a genetically modified cell comprising a deletion in a miR-155 host gene or a fragment thereof.

In some embodiments, the deletion is in exon 1, exon 2, exon 3, or the transcriptional start site of the miR-155 host gene.

In some embodiments, the genetically modified cell is engineered using a method comprising introducing into the cell a CRISPR/Cas endonuclease (Cas)9 system with one or more CRISPR/Cas guide RNAs, wherein the one or more guide RNAs target the miR-155 host gene or a fragment thereof.

In some embodiments, the one or more guide RNAs target exon 1, exon 2, exon 3, intron 1, intron 2, or the transcriptional start site of the miR-155 host gene.

In some embodiments, the guide RNA comprises a polynucleotide sequence at least 80% identical to one of SEQ ID NOs: 1-14. In some embodiments, the CRISPR/Cas9 system comprises a first guide RNA and a second guide RNA. In some embodiments, the first guide RNA comprises a polynucleotide sequence at least 80% identical to SEQ ID NO: 11 or 13 and the second guide RNA comprises a polynucleotide sequence at least 80% identical to SEQ ID NO: 12 or 14. In some examples, the target sequence of the one or more CRISPR/Cas guide RNAs comprises a polynucleotide sequence at least 80% identical to SEQ ID NOs: 35-39.

In some embodiments, the cell is a T cell or a stem cell. In some embodiments, the T cell is a primary T cell, a T cell line, a chimeric antigen receptor (CAR)-T cell, a tumor infiltrating lymphocyte, an effector T cell, a memory T cell, a TEMRA (terminally differentiated effector memory T cell), or a stem cell-like memory T cell.

In some aspects, disclosed herein is a method for treating or preventing graft-versus-host disease in a subject, comprising administering to the subject a therapeutically effective amount of the genetically modified cell of any preceding aspect.

In some embodiments, the cell is a T cell. In some embodiments, the T cell is a primary T cell, a T cell line, a chimeric antigen receptor (CAR)-T cell, a tumor infiltrating lymphocyte, an effector T cell, a memory T cell, a TEMRA, or a stem cell-like memory T cell. In some embodiments, the T cell is a tumor-specific T cell.

In some embodiments, the T cell is cultured ex vivo for at least 2 days prior to the administration. In some embodiments, the T cell is cultured ex vivo for at least 7 days prior to the administration. In some embodiments, the T cell is cultured with IL-2. In some embodiments, the T cell is cultured with IL-2, IL-7, IL-15 or any combination thereof. In some embodiments, the T cell is derived from the subject.

Also disclosed herein is a method of creating a genetically modified T cell comprising a deletion in a miR-155 host gene, said method comprising:
obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.

In some embodiments, the T cell is cultured ex vivo for at least 2 days prior to the introduction of the CRISPR/Cas endonuclease (Cas)9 system. In some embodiments, the T cell is cultured ex vivo for at least 7 days prior to the introduction of the CRISPR/Cas endonuclease (Cas)9 system.

Also disclosed herein is a method for preventing or treating acute graft-versus-host disease in a subject, comprising administering to the subject a genetically modified T cell comprising a deletion in a miR-155 host gene, wherein the genetically modified T cell is created by a method comprising:
obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.

### BRIEF DESCRIPTION OF THE DRAWINGS

The accompanying figures, which are incorporated in and constitute a part of this specification, illustrate several aspects described below.

FIGS. 1A-1B show that miR-155 expression is up-regulated in mouse and human with aGVHD. FIG. 1A shows transplant schema. FIG. 1B shows relative miR-155 expression in CD4+CD62L− and CD8+CD44+ effector T cells isolated from the spleen of three recipient mice with aGVHD (BM+spleen) or controls (TCD-BM). Results shown represent mean±S.D. of three independent mouse samples, normalized to sno135.

FIGS. 2A-2E show histopathological assessment of human small and large bowel samples from patients with aGVHD (FIGS. 2A-2D) or from control patients (FIG. 2E) who had a bowel biopsy but no pathology was observed. In situ hybridization was performed using a digoxigenin-labeled LNA-modified probe complementary to miR-155 (FIGS. 2A, 2C) or a scramble LNA control probe (FIGS. 2B, 2D). In FIG. 2A strong signal for miR-155 (dark staining) in the inflammatory cells of the lamina propria in the colon tissue, magnification 200×. In FIG. 2C, a section of ileum shows a normal villous to the left side the figure, and the loss of the villi in the rest of the section. Note the strong signal in the inflammatory cells in the area of the damaged villi with the miR-155 probe. Magnification is 200×. FIGS. 2B and 2D show staining for FIGS. 2A and 2C respectively with scramble LNA. FIG. 2E shows normal colon mucosa negative for miR-155 staining. Magnification is 400×.

FIGS. 3A-3C show that genomic deletion of miR-155/BIC confers superior protection against aGVHD compared to locked nucleic acid (LNA) anti-miR-155. FIG. 1B shows lethally irradiated F1 recipients received TCD-BM along with 20*10⁶ splenocytes. Recipients were treated with either scramble (SCR control) or LNA antimir-155, three times weekly via tail vein injection. Survival curve is shown. FIG. 3B shows lethally irradiated F1 recipients received TCD-BM alone or TCD-BM along with 5*10⁶ WT T cells. Recipients of allogeneic T cells were treated with either Scramble (SCR) or Miragen LNA anti miR-155, three times weekly via tail vein infection. Survival curve is shown.

FIGS. 4A-4C show that miR-155 deletion in hematopoietic stem cells does not adversely affect engraftment and maintains robust immune response. Lethally irradiated CD45.1+B6 WT recipients received unfractionated bone marrow cells including T cells (10*10⁶ cells) from either CD45.2+B6 WT mice (n=5) or CD45.2+ BIC/miR-155 KO mice (n=5). Mice were bled every alternate week beginning 3 weeks post-transplant until 17 weeks. FIG. 4A shows that engraftment kinetics were monitored by flow cytometric evaluation of CD45.2+ donor cells. FIGS. 4B-4C show that, at week 17, mice from each cohort were injected i.p. with 5 mg/kg LPS (n=3) or saline (n=2, control). Splenocytes were harvested and intracellular TNFα production by CD45.2+CD4+ T cells analyzed by flow cytometry. Representative contour plots (FIG. 4B) and (FIG. 4C) graphical representation, p<0.001.

FIGS. 5A-5B show design of sgRNA pairs to target MIR155HG. FIG. 5A shows MIR155HG gene locus with exons in blue shaded boxes and promoter region. Yellow shaded box within exon 3 denotes pre-miR-155. Guide RNAs (gRNAs) targeting promoter region are shown in pink while gRNAs targeting miR-155 sequence in the exon 3 junction are shown in purple. FIG. 5B show schema of CRISPR/Cas9 strategy to target MIR155HG in primary human T cells.

FIGS. 6A-6D show validation of sgRNA pairs to target miR-155HG. FIG. 6A shows genomic PCR performed 72 hours after transfection. One representative donor is shown. FIG. 6B shows that ddPCR performed 72 hours after transfection. Data is combined from 4 independent donors, each symbol represents an individual donor (paired t-test, **p<0.0). FIG. 6C shows fold change in miR-155 and MIR155HG expression as measured by quantitative real-time at (FIG. 6C) 72 hours post-transfection and (FIG. 6D) 7 days post-transfection. RNU44 and Hprt were used as normalizers for miR-155 and MIR155HG expression respectively. Data is combined from n=3-4 independent donors. Each symbol represents an individual donor.

FIGS. 7A-7C show CRISPR/Cas9 mediated deletion of miR-155HG in human T cells protects from acute GVHD in a xenogeneic model of disease. NSG mice were irradiated with 50 cGy and injected between 5-10 million CRISPR/Cas9 edited human T cells from the same donor. (n=2 per group in two independent transplant experiments from two different human donors. Number of injected T cells were equal between edited and unedited cohorts from each donor.) FIG. 7A shows that Survival curve from two independent transplants. Log-rank test was used to compare survival, p<0.05. FIG. 7B shows acute GVHD clinical scores (**p<0.01; multiple T-tests). FIG. 7C shows representative images of recipient mice at day 65 post-transplant.

FIGS. 8A-8B show gating strategy for T cell purity, viability and activation. FIG. 8A shows that T cells isolated from healthy donor buffy coat were stained with live/dead stain and antibody against CD3. FIG. 8B shows that T cell were activated with CD3/CD28 Dynabeads for 48 hrs in supplemented media. T cell viability and activation was assessed by flow cytometry using live/dead stain and antibodies against CD25 and CD69.

### DETAILED DESCRIPTION

Disclosed herein are compositions, cells, and methods for the treatment and/or prevention of acute graft-versus-host disease.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc

The phrases “concurrent administration”, “administration in combination”, “simultaneous administration” or “administered simultaneously” as used herein, means that the compounds are administered at the same point in time or immediately following one another.

The phrase “codon optimized” as it refers to genes or coding regions of nucleic acid molecules for the transformation of various hosts, refers to the alteration of codons in the gene or coding regions of nucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA occurs.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

The term “genetically engineered cell” or “genetically modified cell” as used herein refers to a cell modified by means of genetic engineering. The term as used herein “engineered” or “modified” thereof may refer to one or more changes of nucleic acids, such as nucleic acids within the genome of an organism. The term “engineered” or “modified” may refer to a change, addition and/or deletion of a gene. Engineered cells or modified cells can also refer to cells that contain added, deleted, and/or changed genes.

As used herein, the term “graft-versus-host” or “GVH” refers to an immune response of graft (donor) cells against host cells and tissues.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs, or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level so long as the decrease is statistically significant.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

“Therapeutically effective amount” or “therapeutically effective dose” of a composition refers to an amount that is effective to achieve a desired therapeutic result. In some embodiments, a desired therapeutic result is reduction or clearance of a pathogen. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “polypeptide” refers to a compound made up of a single chain of D- or L-amino acids or a mixture of D- and L-amino acids joined by peptide bonds.

The term “nucleic acid” as used herein means a polymer composed of nucleotides, e.g. deoxyribonucleotides or ribonucleotides.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Genetically Modified Cells and Uses Thereof

In some aspects, disclosed herein is a genetically modified cell comprising a deletion in a miR-155 host gene or a fragment thereof.

miRNAs are small non-coding RNAs, with an average 22 nucleotides in length. Most miRNAs are transcribed from DNA sequences into primary miRNAs (pri-miRNAs) and processed into precursor miRNAs (pre-miRNAs) and mature miRNAs. The term “miRNA” herein includes the primary (pri-miRNA), precursor (pre-miRNA) and/or mature forms of the miRNA. The term also includes modified forms (e.g., sequence variants) of the miRNA (e.g., 1 2, 3, 4, 5, or more nucleotides that are substituted, inserted and/or deleted). In representative embodiments, the variant substantially retains at least one biological activity of the wild-type miRNA. The term also includes variants that have been modified to resist degradation within a subject and/or within a cell. The term further includes fragments of a miRNA that substantially retain at least one biological activity of the wild-type miRNA. By “substantially retains” at least one biological activity of the wild-type miRNA means at least about 50%, 60%, 70%, 80%, 90% or more of the biological activity of the wild-type miRNA. The one or more biological activities of miRNA can include any relevant activity, including without limitation, binding activity (e.g., to a target mRNA), prevention and/or treatment of GVHD.

The term “miR-155 host gene” herein refers to DNA sequences encoding miR-155. In some examples, the miR155 host gene described herein is encoded on the + strand of human chromosome 21 from 25,562,048 to 25,575,168 (Gene: ENSG00000234883.7, Transcript: ENST00000659862.2). In some embodiments, the miR-155 host gene comprises at least 80% (e.g., at least about 80%, about 85%, about 90%, about 95%, or about 98%) identical to SEQ ID NO: 45 or a fragment thereof. In some embodiments, the deletion of the genetically modified cell disclosed herein is in exon 1, exon 2, exon 3, or the transcriptional start site of the miR-155 host gene. In some embodiments, the deletion is in exon 3. In some embodiments, the deletion is in the transcriptional start site of the miR-155 host gene. In some embodiments, the deletion is in the promoter of the miR-155 host gene. In some embodiments, the start and end positions of the promoter, introns 1 and 2, and exons 1-3 are those shown in Table 4. In some embodiments, the exon 1 of miR-155 host gene comprises at least 80% (e.g., at least about 80%, about 85%, about 90%, about 95%, or about 98%) identical to SEQ ID NO: 41 or a fragment thereof. In some embodiments, the exon 2 of miR-155 host gene comprises at least 80% (e.g., at least about 80%, about 85%, about 90%, about 95%, or about 98%) identical to SEQ ID NO: 42 or a fragment thereof. In some embodiments, the exon 3 of miR-155 host gene comprises at least 80% (e.g., at least about 80%, about 85%, about 90%, about 95%, or about 98%) identical to SEQ ID NO: 43 or a fragment thereof. In some embodiments, the pre-miR-155 coding host gene sequence comprises at least 80% (e.g., at least about 80%, about 85%, about 90%, about 95%, or about 98%) identical to SEQ ID NO: 44 or a fragment thereof.

In some embodiments, the genetically modified cell is engineered using a method comprising introducing into the cell a CRISPR/Cas endonuclease (Cas)9 system with one or more CRISPR/Cas guide RNAs, wherein the one or more guide RNAs target the miR-155 host gene or a fragment thereof.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

The terms “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 by sequence within the guide RNA that specifies the target site and may be used interchangeably with the term “guide” or “spacer”. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences.

In some embodiments, the one or more guide RNAs target exon 1, exon 2, exon 3, intron 1, intron 2, or the transcriptional start site of the miR-155 host gene.

In some embodiments, the CRISPR/Cas endonuclease (Cas)9 system comprises two CRISPR/Cas guide RNAs. In some embodiments, the first and the second guide RNAs targets the transcriptional start site or the promoter of the miR-155 host gene. In some embodiments, the first guide RNA targets a region upstream of the 5′-end of exon 3 (e.g., intron 2) and the second guide RNA targets exon 3.

In some embodiments, the guide RNA comprises a polynucleotide sequence at least about 80% (e.g., at least about 80%, about 85%, about 90%, about 95%, or about 98%) identical a sequence selected from SEQ ID NOs: 1-14. In some embodiments, the first guide RNA comprises a polynucleotide sequence at least 80% (e.g., at least about 80%, about 85%, about 90%, about 95%, or about 98%) identical to SEQ ID NO: 11 or 13 and the second guide RNA comprises a polynucleotide sequence at least 80% (e.g., at least about 80%, about 85%, about 90%, about 95%, or about 98%) identical to SEQ ID NO: 12 or 14.

In some embodiments, the target sequence of the one or more CRISPR/Cas guide RNAs comprises a polynucleotide sequence at least 80% (e.g., at least about 80%, about 85%, about 90%, about 95%, or about 98%) identical to SEQ ID NOs: 35-39. In some embodiments, the promoter targeting sgRNA binding site comprises the sequence of SEQ ID NOs: 35 or 37. In some embodiments, the promoter targeting sgRNA binding site comprises the sequence of SEQ ID NOs: 36 or 37. In some embodiments, the promoter targeting sgRNA binding site comprises the sequence of SEQ ID NOs: 38 or 39.

In some embodiments, the cell is a T cell or a stem cell. In some embodiments, the cell is a T cell. In some embodiments, the cell is a stem cell.

“Stem cells” herein refer to pluripotent cells which are capable of giving rise to indefinitely more cells of the same type, and from which certain other kinds of cell arise by differentiation. In some embodiments, a stem cell has an increased expression of CD34 on its cell surface. In some embodiments, the stem cell is a hematopoietic stem cell.

In some embodiments, the T cell is a primary T cell, a T cell line, a chimeric antigen receptor (CAR)-T cell, a tumor infiltrating lymphocyte, an effector T cell, a memory T cell (e.g., CD45RA^{lo}CD45RO^{hi}CD62L^{hi} central memory T cell and CD45RA^{lo}CD45RO^{hi}CD62L^{lo} effector memory T cell), a TEMRA (terminally differentiated effector memory T cell) (CD45RA^{hi}CD45RO^{lo}CD62L^{lo}), or a stem cell-like memory T cell (CD45RA^{hi}CD45RO^{lo}CD62L^{lo}). In some embodiments, the T cell is a tumor-specific T cell. In some embodiments, the T cell is an activated T cell.

In some embodiments, the T cell is a human T cell.

In some aspects, disclosed is a pharmaceutical composition comprising the genetically modified cell disclosed herein and a pharmaceutically acceptable carrier.

Also disclosed herein is a composition comprising the CRISPR/Cas9 system and the one or more CRISPR/Cas guide RNAs disclosed herein.

In some aspects, disclosed herein is a method of creating a genetically modified T cell comprising a deletion in a miR-155 host gene, said method comprising:
obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.

In some embodiments, the deletion is in exon 1, exon 2, exon 3, or the transcriptional start site of the miR-155 host gene.

In some embodiments, the genetically modified cell is engineered using a method comprising introducing into the cell a CRISPR/Cas endonuclease (Cas)9 system with one or more CRISPR/Cas guide RNAs, wherein the one or more guide RNAs target the miR-155 host gene or a fragment thereof.

In some embodiments, the one or more guide RNAs target exon 1, exon 2, exon 3, intron 1, intron 2, or the transcriptional start site of the miR-155 host gene.

In some embodiments, the guide RNA comprises a polynucleotide sequence at least 80% identical to one of SEQ ID NOs: 1-14. In some embodiments, the CRISPR/Cas9 system comprises a first guide RNA and a second guide RNA. In some embodiments, the first guide RNA comprises a polynucleotide sequence at least 80% identical to SEQ ID NO: 11 or 13 and the second guide RNA comprises a polynucleotide sequence at least 80% identical to SEQ ID NO: 12 or 14. In some examples, the target sequence of the one or more CRISPR/Cas guide RNAs comprises a polynucleotide sequence at least 80% identical to SEQ ID NOs: 35-39.

In some embodiments, the cell is a T cell or a stem cell. In some embodiments, the T cell is a primary T cell, a T cell line, a chimeric antigen receptor (CAR)-T cell, a tumor infiltrating lymphocyte, an effector T cell, a memory T cell, a TEMRA, or a stem cell-like memory T cell.

In some embodiments, the method further comprises culturing the T cell ex vivo prior to the introduction of the CRISPR/Cas endonuclease (Cas)9 system. In some embodiments, the T cell is cultured ex vivo for at least 2 days (e.g., at least 2 days, 3 days, 4 days, 5 days, 6 days, 7 days, 8 days, 9 days, or 10 days) prior to the introduction of the CRISPR/Cas endonuclease (Cas)9 system. In some embodiments, the T cell is culture ex vivo for at least 7 days prior to the introduction of the CRISPR/Cas endonuclease (Cas)9 system. T cell can be cultured with, including, for example, IL-2, anti-CD3 antibody, and/or anti-CD28 antibody. In some embodiments, the T cell is derived from the subject.

In some aspects, disclosed herein is a method for treating or preventing graft-versus-host disease (GVHD) in a subject, comprising administering to the subject a therapeutically effective amount of the genetically modified cell as disclosed herein.

As used herein, the term “graft-versus-host disease” or “GVHD” refers to a condition, including acute and chronic, resulting from transplanted (graft) cell effects on host cells and tissues resulting from an allogeneic hematopoietic cell transplant. In other words, donor immune cells infused within the graft or donor immune cells that develop from the stem cells, may see the patient's (host) cells as foreign and turn against them with an immune response. As examples, patients who have had a blood or marrow transplant from someone else are at risk of having acute GVHD. Even donors who are HLA-matched with the recipient can cause GVHD because the donor cells can potentially also make an immune response against minor antigen differences in the recipient. Acute graft-versus-host disease (GVHD) is a disorder caused by donor immune cells in patients who have had an allogeneic marrow or blood cell transplantation. The most commonly affected tissues are skin, intestine and liver. In severe cases, GVHD can cause blistering in the skin or excessive diarrhea and wasting. Also, inflammation caused by donor immune cells in the liver can cause obstruction that causes jaundice. Other tissues such as lung and thymus may also become affected. The diagnosis is usually confirmed by looking at a small piece of skin, liver, stomach or intestine with a microscope for observation of specific inflammatory characteristics. The symptoms of acute GVHD further comprises an increase of white blood cell counts and proinflammatory cytokine levels. The symptoms of acute GVHD usually begins within the first 3 months after the transplant. In some cases, it can persist, come back or begin more than 3 months after the transplant. In some examples, the compositions and methods disclosed herein are used for treatment and/or prevention of acute GVHD.

The cells and methods disclosed herein for preventing and/or treating acute GVHD can be a prevention and/or treatment of one or more of blistering in the skin, skin rashes, abdominal cramps, excessive diarrhea, inflammation in the liver, intestine, lung, thymus, jaundice, and/or nausea.

In some embodiments, the cell is a T cell. Accordingly, in some aspects, disclosed herein is a method for treating or preventing acute graft-versus-host disease in a subject, comprising administering to the subject a therapeutically effective amount of the T cell disclosed herein. In some embodiments, the T cell is a primary T cell, a T cell line, CAR-T, tumor infiltrating lymphocytes, effector T cell, memory T cell, TEMRA, or stem cell-like memory T cell. In some embodiments, the T cell is a tumor-specific T cell.

In some embodiments, the T cell is cultured ex vivo for at least 2 days prior to the administration (e.g., the T cell is cultured ex vivo for at least 2 days, 3 days, 4 days, 5 days, 6 days, 7 days, 8 days, 9 days, or 10 days prior to the administration). In some embodiments, the T cell is culture ex vivo for at least 7 days prior to the administration. T cell can be cultured with, including, for example, IL-2, anti-CD3 antibody, and/or anti-CD28 antibody. In some embodiments, the T cell is derived from the subject.

Also, in some aspects, disclosed herein is a method of treating or preventing graft-versus-host disease (GVHD) in a subject, comprising
a) obtaining T cells and bone marrow cells/stem cells;
b) introducing into the T cell a CRISPR/Cas endonuclease (Cas)9 system with one or more CRISPR/Cas guide RNAs, wherein the one or more guide RNAs target the miR-155 host gene or a fragment thereof;
c) administering to the subject an effective amount of the T cells and the bone marrow cells or stem cells to the subject.

Also disclosed herein is a method for preventing or treating acute graft-versus-host disease in a subject, comprising administering to the subject a genetically modified T cell comprising a deletion in a miR-155 host gene, wherein the genetically modified T cell is created by a method comprising:
to obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.

In some embodiments, the method further comprises culturing the T cell ex vivo prior to or after step b. In some embodiments, the T cell is cultured ex vivo for at least 2 days (e.g., at least 2 days, 3 days, 4 days, 5 days, 6 days, 7 days, 8 days, 9 days, or 10 days) prior to or after step b. In some embodiments, the T cell is culture ex vivo for at least 7 days prior to or after step b. T cell can be cultured with, including, for example, a cytokine (e.g., IL-2, IL7, IL-15, or a combination thereof), anti-CD3 antibody, and/or anti-CD28 antibody. In some embodiments, the T cell is culture ex vivo for at least 7 days prior to or after step b. T cell can be cultured with, including, for example, IL-2, anti-CD3 antibody, and/or anti-CD28 antibody. In some embodiments, the T cell is culture ex vivo for at least 7 days prior to or after step b. T cell can be cultured with, including, for example, IL-2, IL-7, IL-15, anti-CD3 antibody, and/or anti-CD28 antibody. In some embodiments, the T cell is derived from the subject.

In some examples, the subject is diagnosed as having a blood cancer (including, for example, Acute leukemia, Chronic leukemia, Hodgkin's lymphoma, Multiple myeloma, Neuroblastoma, or Non-Hodgkin's lymphoma). It should be understood and herein contemplated that the cells and methods disclosed herein are effective in treating a blood cancer in a subject without causing acute graft-versus-host disease.

Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, intravaginal, by inhalation, via an implanted reservoir, or via a transdermal patch, and the like.

In some embodiments, the method of any preceding aspect further comprises administering to the subject a therapeutically effective amount of an additional agent for treating aGVHD. In some embodiments, the additional agent is selected from the group consisting of methotrexate, cyclosporine, tacrolimus, mycophenolate mofetil, sirolimus, corticosteroid, anti-thymocyte globulin, alemtuzumab, and cyclophosphamide.

Dosing frequency for the genetically modified cell disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, dosing frequency for the genetically modified cell disclosed herein includes at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks or once a week. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for genetically modified cell includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is less than about 4 months, less than about 3 months, less than about 2 months, less than about a month, less than about 3 weeks, less than about 2 weeks, or less than less than about a week. In some embodiments, the interval between each administration is constant. For example, the administration can be carried out daily, every two days, every three days, every four days, every five days, or weekly. In some examples, the administration can be carried out every week, every two weeks, or every two months.

At time of allogeneic transplant, or as donor lymphocyte infusions (DLI) to treat relapse post-transplant, the dose of T cells can vary from, for example, from about 1×10⁵ to about 200×10⁸ CD3+ T cells/kg body weight. In some embodiments, the dose of T cells is from about 1×10⁶ to about 200×10⁶ CD3+ T cells/kg body weight. In some embodiments, the dose of T cells is about 1×10⁶ CD3+ T cells/kg body weight, about 2×10⁶ CD3+ T cells/kg body weight, about 3×10⁶ CD3+ T cells/kg body weight, about 5×10⁶ CD3+ T cells/kg body weight, about 10×10⁶ CD3+ T cells/kg body weight, about 20×10⁶ CD3+ T cells/kg body weight, about 50×10⁶ CD3+ T cells/kg body weight, about 80×10⁶ CD3+ T cells/kg body weight, about 100×10⁶ CD3+ T cells/kg body weight, about 150×10⁶ CD3+ T cells/kg body weight, or about 200×10⁶ CD3+ T cells/kg body weight.

### EXAMPLES

The following examples are set forth below to illustrate the compositions, cells, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

Acute graft-versus-host disease (aGVHD) is a frequent and lethal complication of allogeneic hematopoietic stem cell transplantation (allo-HSCT) in which donor T cells destroy HLA mismatched host tissues by secreting inflammatory cytokines (TNF-α and IFN-γ) and/or inducing direct cytotoxic cellular responses. T cells in the donor graft are essential for aGVHD pathogenesis, however, donor T cells are also critical to eradicate residual leukemia and prevent relapse, in a process called graft versus leukemia (GVL) effect. Current aGVHD treatments are broadly immunosuppressive, and regimens successful in preventing aGVHD come at the cost of increased risk of relapse (due to loss of GVL effect) and/or infections. Thus, there is an urgent need for novel therapies that can restrain overt T cell activation preventing aGVHD without compromising on relapse.

MicroRNAs (miR) play critical roles in the development and function of the immune system. miR-155 expression is up-regulated in T cells of mice developing lethal aGVHD following WIC disparate bone-marrow (BM) transplant. Mice receiving allogeneic miR-155−/− donor T cells do not exhibit GVHD, have lower serum TNT-α levels and improved survival compared to mice that received miR-155 sufficient lymphocytes. Importantly, miR-155−/− cells mounted GVL responses similar to wild type T cells in murine models. Relevant to human disease, dramatic up-regulation of miR-155 was found in clinical specimens from patients with GI tract aGVHD. A follow-up study showed that miR-155 impacts expansion and migration of donor T cells to GVHD target organs such as liver and GI tract thereby modulating aGVHD pathogenesis. These data indicate a role for miR-155 in the modulation of GVHD, and point to miR-155 as a target for therapeutic intervention in this disease. Treatment of recipient mice after allogeneic transplants with antagomiR-155 decreases aGVHD; however, efficiency of targeting miR-155 was low and responses not robust. Therefore, there is an unmet need to target miR-155 using different strategies.

The CRISPR (clustered, regularly interspaced, short palindromic repeats)/Cas9 (CRISPR-associated protein 9) system uses a single protein, Cas9 that is guided by single-guide RNA to target DNA through Watson-Crick base pairing. Using CRISPR-Cas9 strategy, high genome editing performance has been shown in human hematopoietic cells, including stem cells and T cells. Recently, a recent work reported a phase 1 clinical trial to assess safety and feasibility of CRISPR-Cas9 gene editing of T cells. These modified T cells were well tolerated and have durable engraftment and open avenues to study CRISPR-engineered immunotherapies.

Disclosed herein is a strategy to treat aGVHD by deleting genomic miR-155HG from donor T cells using CRISPR/CAS9 system. Removing genomic miR-155 can prevent aGVHD without affecting GVL effects.

Assessment of CRISPR/CAS9 efficiency in deletion of genomic miR-155 and off-target effects by next-generation sequencing (NGS). Guide RNAs are transfected along with Hi-Fi CAS9 system first into K562 cells (for optimization) and then human cells using nucleoporation. Whether guides are efficient in removing miR-155HG and assess off-target effects is confirmed by performing NGS.

Evaluation of CRISPR/Cas9 engineered miR-155 KO human T cells in preclinical mouse models of GVHD and GVL. In vivo studies of CRISPR/Cas9 engineered miR-155 KO human T cells are conducted in a xenogeneic model of aGVHD and aGVHD incidence, severity and outcome are evaluated. Following this, the GVL-sparing effect of CRISPR/Cas9 engineered miR-155 KO human T cells is evaluated in a xenogeneic model of GVL.

The instant disclosure shows a strategy to prevent aGVHD while having positive GVL effects. This method can be applied to stem cell transplant and eliminate immunosuppression and related complications from these treatments.

### Example 2

Acute GVHD and related complications is the major cause of non-relapse mortality in patients receiving an allogeneic hematopoietic stem cell transplant (allo-HSCT). Despite the use of standard GVHD prophylaxis regimens such as calcineurin inhibitors and other agents, 30-75% of allo-HCT patients eventually develop aGVH. A majority of aGVHD patients (40-60%) exhibit steroid resistance, associated with poor prognosis with only 5-25% long-term survival. Thus, aGVHD is an unmet clinical need for new therapeutic options.

miR-155 is upregulated in T cells of mice developing aGVHD following allogeneic transplant. Mice receiving allo-transplant from miR-155−/− donors did not develop aGVHD but still mounted an appropriate graft versus leukemia (GVL) response. Relevant to human disease, dramatic up-regulation of miR-155 was also found in clinical specimens from patients with GI aGVHD. Targeting miR-155 using anti-sense oligonucleotides showed very little improvement in survival of mice receiving an allo-transplant.

### Example 3

Currently, prevention of aGVHD is through pharmacological immunosuppression or T cell depletion. However, T cells in the donor graft perform important functions such as facilitating HSC engraftment and providing protection against opportunistic pathogens. Using CRISPR/Cas9 technology to delete genomic DNA corresponding to non-coding RNA (BIC/miR-155) is an innovative strategy to produce engineered miR-155 KO human T cells that can prevent GVHD yet maintain response against infectious pathogens that can yield superior outcomes post-transplant compared to traditional approaches.

Biology and clinical significance of Acute Graft-Versus-Host Disease (aGVHD): More than 8000 patients receive an allo-HSCT annually in the US alone as a cure for hematologic malignancies and other primary bone marrow disorders. However, the major barrier for the success of allo-HCT is the high incidence of aGVHD and its associated morbidity and mortality. The pathogenesis of aGVHD involves recognition of host antigens by donor T cells followed by expansion, migration and finally end-organ damage due to combination of inflammatory cytokine secretion and direct cytotoxic effects. Acute GVHD is clinically characterized by damage to the skin, liver and gastrointestinal (GI) tract. The incidence and severity of aGVHD depends on the degree of histocompatibility between donor and recipient, the donor graft source (peripheral blood or bone marrow), recipient's age, and GVHD prophylactic regimen.

Limitations of current therapies: There are several different immunosuppressive drug regimens currently used to prevent aGVHD after allo-HSCT. The most common regimens include drug combinations using methotrexate, calcineurin inhibitors (cyclosporine or tacrolimus), cyclophosphamide and micophenolate mofetil. However, despite aGVHD prophylaxis, aGVHD still occurs frequently in about 30 to 70% of all recipients of allo-HSCT. The standard first line treatment for aGVHD is high-dose of corticosteroids. However, about 50% of patients with aGVHD are not responsive to this therapy and have a very high non-relapse mortality. Among the different treatment strategies for steroid refractory (SR) aGVHD include JAK/STAT inhibition, anti-IL-6 and anti-TNF-α monoclonal antibodies (mAbs), T-cell trafficking blockade, immunomodulation with alpha-1 antitrypsin, and extracorporeal photopheresis. These therapies have shown varying levels of response in clinical trials. Recently the FDA approved the oral JAK1/JAK2 inhibitor, ruxolitinib, for SR-aGVHD in combination with steroids based on a small open-label, single-arm, multicenter study of ruxolitinib that enrolled 49 patients with SR-aGVHD occurring after allo-HSCT. In this study, the overall response rate (ORR) was of 55% and the complete response (CR) was 27% by day 28 after treatment with ruxolitinib in combination with steroids (NCT02953678). The therapeutic potential of other agents such as anti-thymocyte globulin and anti-CD52 mAb is hindered by the increased risk of lethal infections and/or relapse. Thus, there is a need for novel, effective therapies with improved tolerability to prevent and treat aGVHD.

miR-155 is upregulated in donor T cells and cause aGVHD in recipients. miR-155 expression is up-regulated in T cells of mice developing lethal aGVHD following an MHC disparate BM transplant. Mice that received allogeneic miR-155−/− donor lymphocytes did not exhibit lethal GVHD, had lower serum TNF-α levels and improved survival as compared to mice that received allogeneic WT donor lymphocytes. In contrast, mice that received miR-155 overexpressing T cells displayed accelerated and lethal aGVHD. Importantly, miR-155−/− cells were able to mount graft versus leukemia (GVL) responses similar to recipients of wild type cells in GVL murine models. Relevant to human disease, dramatic up-regulation of miR-155 was also found in clinical specimens from patients with GI tract aGVHD. Collectively, these data indicate a role for miR-155 in the modulation of GVHD, and point to miR-155 as a novel target for therapeutic intervention.

Genome editing of T cells. Gene editing of primary human T cells is a novel strategy that can enhance cellular therapeutic approaches. The CRISPR (clustered, regularly interspaced, short palindromic repeats)/Cas9 (CRISPR-associated protein 9) system uses a single protein, Cas9 that is guided by single-guide RNA to target DNA through Watson-Crick base pairing. Earlier approaches to gene editing involved viral delivery or electroporation mediated transfection of gRNA/Cas9. However, these approaches had major drawbacks including low efficiency and high toxicity. Recent improvements include the use of electroporation of Cas9 ribonucleoprotein (RNPs) that contain a complex of recombinant Cas9 protein with either in-vitro transcribed or synthetic single guide RNA (sgRNA). This approach to transfect activated human T cells has resulted in high efficiency (50-90%) in multiple targets such as CXCR4, CCR5. Additionally, the use of high fidelity Cas9 (HiFi Cas9)-guided CRISPR system has been shown to efficiently engineer primary murine and human T cells. Use of HiFi Cas9 significantly reduces off target effects without affecting on-target efficiency. Recently, a phase 1 clinical trial assessed the safety and feasibility of CRISPR-Cas9 gene editing of T cells in three patients with advanced cancer. These modified T cells were well tolerated and have durable engraftment.

In this example, high fidelity Cas9 (Hi-FiCAS9)-guided CRISPR genome editing is used to knockout genomic miR-155 in human donor T cells prior to allo-HSCT. CRISPR/Cas9 engineered miR-155 KO human T cells protects recipients of an allo-HSCT from aGVHD while maintaining a robust GVL response.

### Example 4

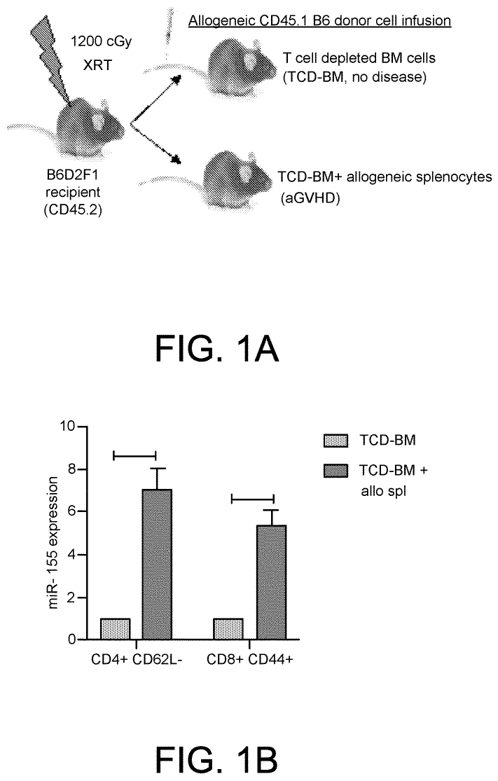

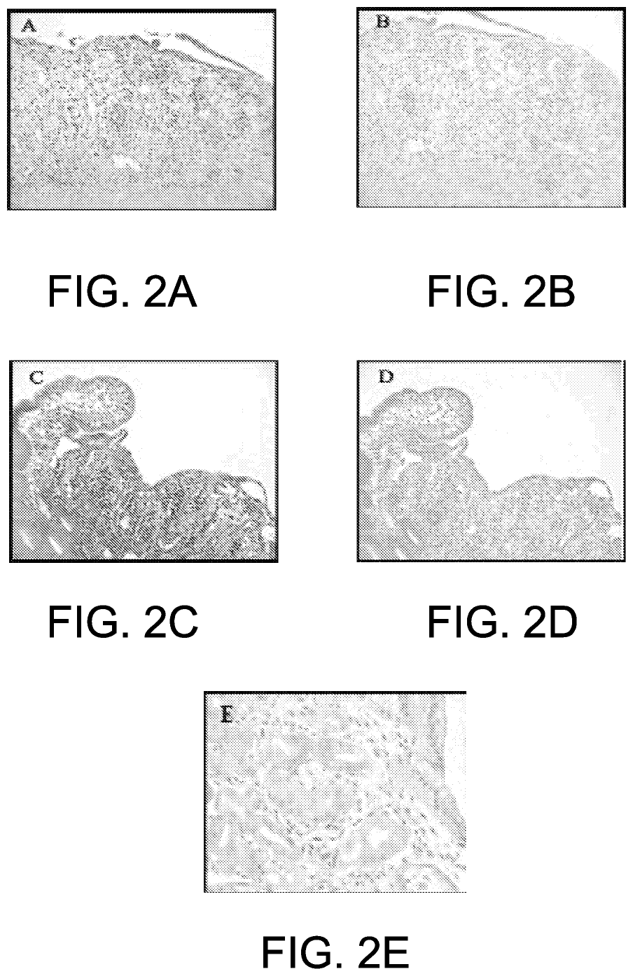

miR-155 is upregulated in mouse and human aGVHD. To investigate the role of miR-155 during aGVHD, the well-established B6 into B6D2F1 haploidentical model of transplant was used. T cells were isolated from irradiated F1 mice that received either T cell depleted BM cells (TCD-BM, no disease) or TCD-BM+B6 allogeneic splenocytes (resulting in aGVHD) 25 days post-transplant. Both CD4+ and CD8+ T cell populations isolated from mice with aGVHD exhibited increased miR-155 expression (6.5 and 5-fold, respectively) with respect to the same cell populations obtained from the controls (FIGS. 1A, 1B). To establish the relevance of the mouse findings to the human system, miR-155 expression was measured using LNA-based in situ hybridization from clinically and histologically confirmed small and large bowel biopsies of aGVHD patients and healthy controls (FIG. 2). A strong up-regulation of miR-155 expression in the inflammatory T cells was observed from all patients with small and large bowel aGVHD, while miR-155 expression was absent in normal bowel (FIG. 2).

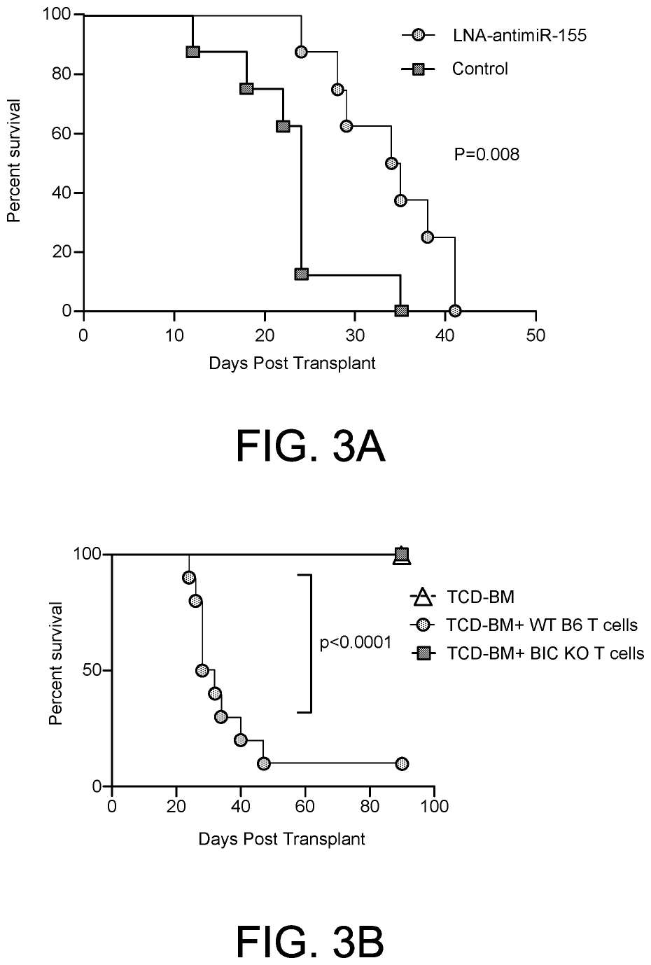

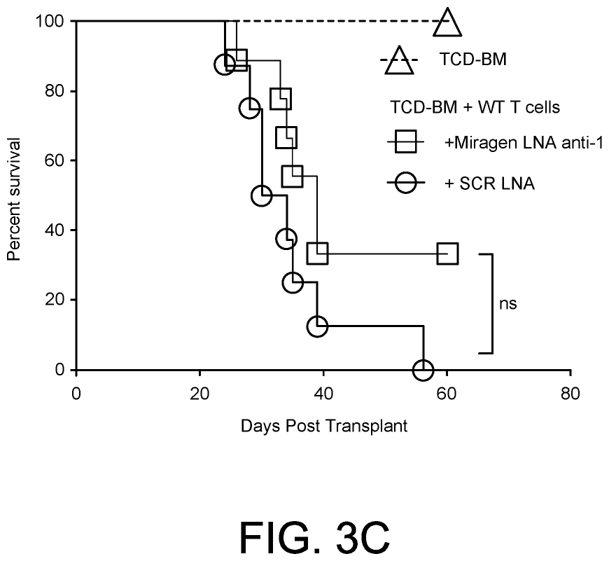

Genomic deletion of miR-155/BIC confers superior protection against aGVHD compared to locked nucleic acid (LAN) anti miR-155 targeting strategies: Treating B6D2F1 recipient mice transplanted with allogeneic B6 T cells with LNA anti-miR-155 was not as effective in preventing aGVHD compared with B6D2F1 recipient mice transplanted with allogeneic miR-155−/− T cells (FIG. 3A). To further optimize these results, the well-established B6 into B6D2F1 (parent into F1) haploidentical model of transplant was used. Lethally irradiated B6D2F1 mice received T cell depleted BM cells (TCD-BM) along with allogeneic B6 WT or miR-155−/− T cells (FIG. 3B). In a separate experiment, F1 mice that received TCD-BM along with allogeneic B6 WT T cells were treated with either saline or LNA anti-miR-155. While there was some improvement in survival of mice receiving anti miR-155 (FIG. 3C), the data clearly show that genomic deletion of miR-155 confers superior protection against aGVHD (FIG. 3B vs. FIGS. 3A and 3C). The results using LNA antimiR-155 did not improve after using different doses, schedules and compounds, including the current LNA antimiR-155 in clinical trials (Cobomarsen from Miragen) as seen in FIG. 3C. The efficiency of knock down was never greater than 50%. Thus, novel strategies to knock down miR-155 are needed.

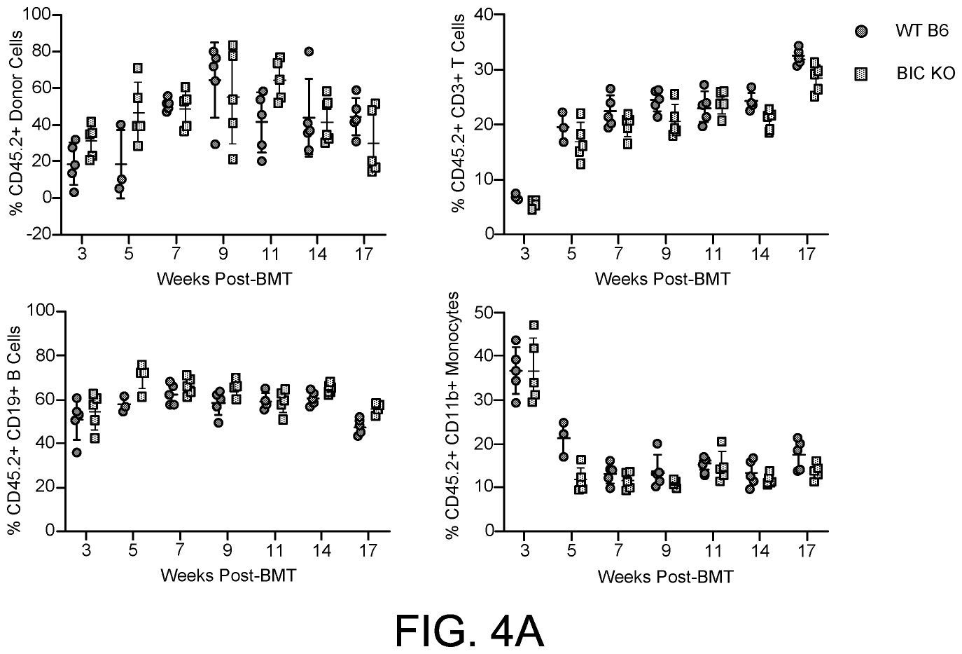

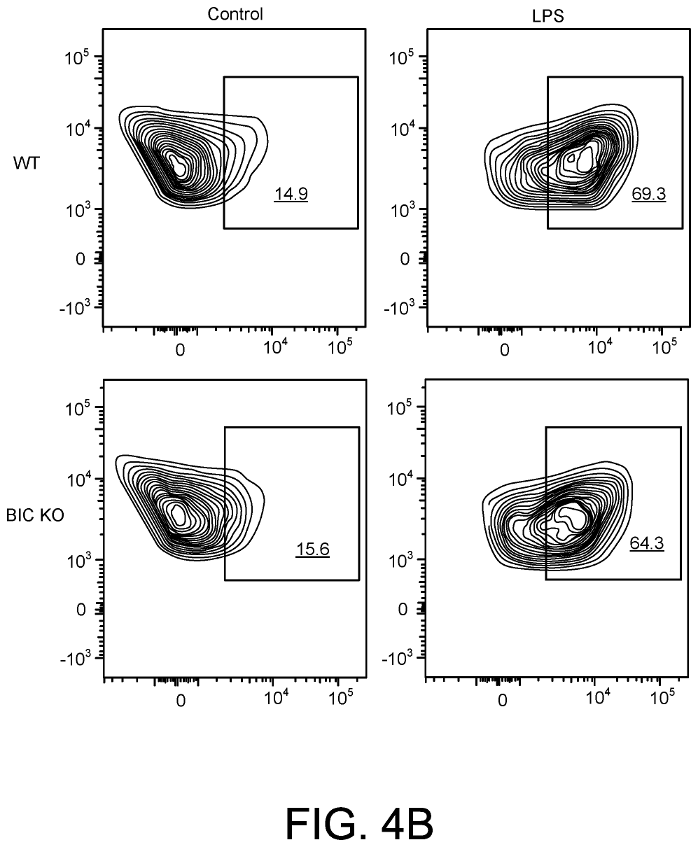

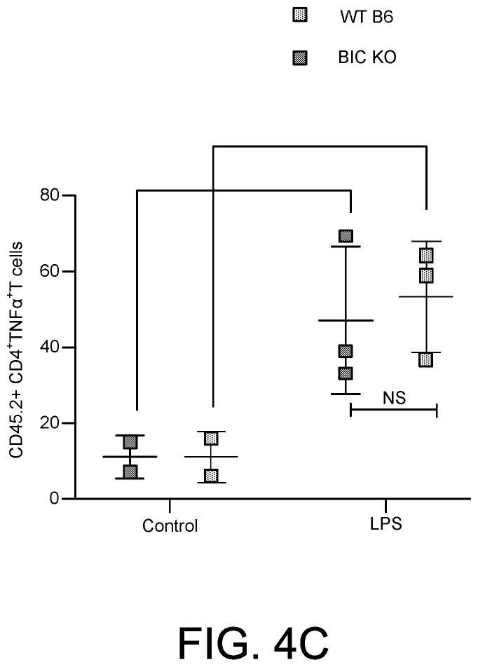

MiR-155 deletion does not adversely affect engraftment and maintains robust immune response. Donor stem cell engraftment is critical to the success of an allogeneic HSCT; therefore, the effect of miR-155 genetic ablation on donor cell engraftment was evaluated. To do so, CD45.1 B6 congenic mice (hereafter referred to as BoyJ) were lethally irradiated and the mice was transplanted with unfractionated bone marrow cells (10*10⁶ cells) including T cells from either CD45.2+B6 WT mice (n=5) or CD45.2+ BIC/miR-155 KO mice (n=5). Engraftment kinetics were monitored by flow cytometric evaluation of CD45.2+ donor cells. B6 WT and miR-155 KO BM cells demonstrated comparable re-emergence of donor populations including CD3+ T cells, CD19+ B cells and CD14+ monocytes that was sustained through week 17 post-transplant (FIG. 4A). At the end of the study, mice from each cohort were injected with either LPS mimicking a bacterial infection, or saline control.

T cells from recipients of both B6 WT and miR-155 KO BM had low to no intracellular TNF-α production in response to control (saline) stimulation. In contrast, T cells from recipients of both B6 WT and miR-155 KO BM displayed similar effector response to LPS stimulation as measured by intracellular TNF-α production (FIGS. 4B, 4C). Altogether, these data show the conclusion that genomic deletion of miR-155 does not adversely affect engraftment and maintains robust effector T cell responses to pathogenic stimuli.

The evidence herein shows that miR-155/BIC gene is upregulated in donor T cells, modulates aGVHD in recipients and its deficiency did not impact beneficial GVL responses. Therefore, genetical manipulation of the primary human donor T cells can be used to prevent aGVHD. Additionally, absence of genomic BIC/miR-155 does not negatively impact engraftment and allows for robust immune response against pathogenic antigens such as (LPS). Therefore, genetically ablating miR-155/BIC gene using CRISPR/HiFi Cas9 system can result in considerable reduction of aGVHD severity and retain GVL effects without negatively impacting engraftment and/or infectious immunity.

### Example 5

### Assessment of CRISPR/CAS9 Efficiency in Deletion of Genomic miR-155 and Off-Target Effects by Next-Generation Sequencing (NGS)

Assessment of CRISPR/CAS9 efficiency and analysis of off-target effects by NGS in K562 cells. Experiment 1: For genomic deletion, it is recommended to have 3-5 distinct gRNAs per loci to ensure efficient results. Therefore, ten gRNAs (using published and web-based algorithms) were generated to target miR-155HG. The miR-155HG specific guides (gRNA) and controls were transfected along with the HiCAS9 system into K562 cells using nucleoporation (AMAXA, solution V). The reason of using K562 cells is that efficiency of transfection is very high (>90%) so there are no confounding factors with delivery efficiency and the CRISPR/Cas9 and gRNA efficiency can be assessed in cutting the genomic DNA. DNA is obtained at several time points (24, 48 and 72 hours) and a quantitative RT-PCR for miR-155HG is performed. Bands are cut from the gel and submitted for Sanger sequencing to confirm genomic deletion. Appropriate negative controls such as nontargeting gRNAs (commercially available) are included.

Experiment 2: Evaluation of CRISPR/CAS9 targeting efficiency using Next Generation Sequencing (NGS). i) GUIDE-Seq: Off-target sites cleaved by Cas9 are detected by performing Genome-wide Unbiased Identification of Double-stranded breaks Enabled by Sequencing (GUIDE-Seq) on samples transfected with Cas9 enzyme and gRNAs. GUIDE-Seq is an unbiased approach to detect off-target breaks using a double-stranded oligodeoxynucleotide (dsODN) to tag cleavage sites. DNA sites that have integrated dsODN are mapped by amplification followed by NGS and ii) Quantitative assessment of indel frequency by Tracking of Indels by DEcomposition (TIDE sequencing): to evaluate the efficiency of the CRISPR knockouts. TIDE sequencing quantifies genome editing efficiency and identifies indels and their frequency in cell populations. TIDE aligns the sequence of the gRNA with the unedited control sequence to determine where Cas9 cleavage can occur. The regions upstream of break site is compared between control sequence and experimental samples to determine whether breakage has occurred. Finally, an appropriate software is used to generate a series of peaks that correspond to the abundance of differing nucleotides in the sequence. These results reveals specific indels allowing the determination of the accuracy and efficacy of the selected gRNAs. The guides are highly efficient in removing genomic DNA containing the miR-155 region with minimal off-target effects.

Assessment of CRISPR/CAS9 efficiency and specificity in human T cells. After the confirmation that the designed gRNAs are efficient with no off-target effects of relevance in K562 cells, the efficiency and specificity of the CRISPR/CAS9 system in human T cells are validated.

Experiment 1. T cells are isolated by EasySep T Cell Isolation Kit (StemCell Technologies) from healthy donor PBMCs (Versiti). Purity of cells is evaluated and confirmed to be greater than >95% before proceeding to the next step. Human T cells are activated and expanded according to established and published protocols for CRISPR/Cas9 application in human T cells. Briefly, T cells are expanded using the T Cell Activation and Stimulation Kit (Miltenyi Biotec). Activation status is confirmed by flow cytometric evaluation of CD25, CD69 prior to nucleofection. Nucleofection of flow-sorted activated primary human T cells is carried out using Amaxa Nucleofector (program for nucleofection of primary T cells, pulse code EH-115 and buffer P3). Deletion of genomic miR-155HG is confirmed by Sanger sequencing as described above.

Experiment 2. Off-target effects are assessed by Guide-Seq and TIDE-Seq as described above.

CRISPR/Cas9 engineered human T cells that carry a deletion of miR-155 genomic region are generated. The functional capacity of the CRISPR/Cas9 engineered miR-155 KO T cells is evaluated using preclinical mouse models of GVHD and GVL.

### Evaluation of CRISPR/Cas9 Engineered T Cells in Preclinical Mouse Models of GVHD and GVL

The main goal of an allogeneic transplant is to induce a donor anti-leukemia response to eliminate residual leukemia/lymphoma in the recipient; any therapeutic strategy targeting aGVHD must not abrogate the GVL response. Identification of agents capable of driving GVL while minimizing GVHD remains an unmet need. In this experiment, the GVHD-reducing and GVL-sparing effects of CRISPR/Cas9 engineered miR-155 KO human T cells are evaluated using in vivo models of aGVHD and GVL.

Xenogeneic aGVHD mouse model: NSG mice are irradiated (50 cGy) and injected with CRISPR/Cas9 miR-155 deleted 5-10×10⁶ human T cells or control T cells transfected with a non-targeting gRNA. T cells for this experiment are generated as described above. Unmanipulated T cells can serve as an additional experimental control. Mice are monitored for survival and clinical signs of GVHD. GVHD is also assessed by detailed histopathology analysis of H&E stained liver and gut tissues using a previously reported scoring system with a range of 0 (absence of signs of GVHD) to 4 (maximal GVHD damage). An experienced pathologist reads all the samples in a blinded fashion. One cohort is used for survival analysis while a different cohort is used for histopathological analysis at day 20, this timepoint is one at which aGVHD is evident in these models but it is before mice start dying from disease.

Clinical scoring of aGVHD: Briefly, this scoring system incorporates five clinical parameters; weight loss, posture (hunching), activity, fur texture and skin integrity. Individual mice are graded (in a scale from 0 to 10) three times a week and mice that reach a GVHD score of ≥7 are euthanized and tissues harvested.

Ex-vivo studies: Mice are bled weekly, and human CD45, CD3, CD4, Foxp3, CD8 T cells are enumerated by flow-cytometry. A separate cohort of mice are euthanized around day 20 post-transplant for histo-pathological evaluation of GVHD target tissues (liver, GI tract and skin). Ex-vivo analysis of human T cell proliferation (Ki67), CD4 and CD8 T cell effector function (intracellular cytokines—INF-γ, TNF-α, IL-17, granzyme/perforin, CD107a); T cell skewing (T-bet, ROR-γt, Foxp3); T cell exhaustion (PD-1, PD-L1, Lag-3 Tim-3) is performed by flow cytometry.

The NSG mice that received control T cells (CRSIPR/Cas9 transfected with non-targeting gRNAs or unmanipulated T cells) die by week 5 due to aGVHD as confirmed by histopathological evaluation and clinical observation. NSG mice that receive CRISPR/Cas9 miR-155 deleted T cells show improved survival, lower incidence and severity of aGVHD.

Xenogeneic GVL model: NSG mice are irradiated (50 cGy) and injected with luciferase transduced FLT3-ITD+ MV4-11 AML cell line to evaluate human GVL effect in PBMCs into NSG model of xenogeneic aGVHD/GVL. Presence of luciferase allows for the tracking of tumor persistence in live animals using whole-body imaging. Irradiated NSG recipients are injected with 1) 1*106 luciferase transduced human FLT3-ITD+ MV4-11 cells alone (luc+MV4-11); or 2) luc+MV4-11 along with 5-10×10⁶ CRISPR/Cas9 miR-155 deleted human T cells; or 3) luc+MV4-11 along with 5-10×10⁶ CRISPR/Cas9 non-targeting gRNA transfected control T cells. Recipients are monitored for signs of tumor burden and clinical GVHD. One cohort is used for clinical scoring and survival, while two other cohorts are used for pathology assessment and bioluminescence. Ex-vivo analysis is performed as described earlier.

NSG mice that received only MV4-11 cells have widespread leukemic infiltration of tissues and die from leukemia by week 3-4. Mice that received MV4-11 cells along with CRISPR/Cas9 non-targeting gRNAs human T cells have significantly lower tumor burden by whole body imaging and IHC assessment as compared to cohort 1. 50% or more from this group die by week 4-5, but cause of death is aGVHD and not leukemia. Mice that received MV4-11 cells along with CRISPR/Cas9 miR-155 deleted human T cells (group 2) have minimal tumor burden similar to mice treated with control T cells (group 3) but exhibit prolonged survival because of the lack of aGVHD, indicating that CRISPR/Cas9 miR-155 deleted human T cells retain the ability to recognize and eliminate tumor cells without causing aGVHD.

Statistical Considerations: For in vivo studies, the comparison is between recipients of CRISPR/Cas9 miR-155 deleted T cells vs. control T cells transfected with a non-targeting gRNA. Based on the data shown herein, survival probability of mice engrafted with control T cells at day 90 is 1% vs. 100% in mice treated genomic miR-155 deleted T cells. If assuming that CRISPR-Cas9 genomic deletion results in 50% of mice alive at day 90, n=13 mice per group are needed for 80% power to detect a significant increase in survival relative to control group, using a 1-sided Fisher's exact test at alpha-=0.05. Therefore, at least 13-15 age and sex-matched recipient mice are transplanted in every allo-group and each experiment is repeated 2-3 times for rigor, reproducibility and to obtain statistical significance. This process is repeated for each of the models (GVHD and GVL). Survival curves are calculated using Kaplan-Meier method and differences assessed using log-rank test. Continuous variables such as GVHD clinical score and weight changes are assessed using linear mixed effects models. For ex-vivo analyses, to assess effect of miR-155 deletion on Th1/Th17 phenotype (5 markers: IFN-γ, IL-17, TNF-α, ROR-γt, T-bet), a sample size of n=13 per group has 80% power to detect a 2-fold difference in the expression of phenotypic markers between groups for a target using a conservative α=0.01 (0.05/5) level of significance and CV=0.5.

TABLE 1
gRNA sequences.

| | Guide sequence | |
|---|---|---|
| Guide | (5′ to 3′, PAM | |
| name | not included) | Citation |
| gRNA1 | GUUAAUGCUAAUCGUGAUAG | Wallace et al (2016) |
| | (SEQ ID NO: 1) | |
| gRNA2 | GCUAAUAUGUAGGAGUCAGU | Wallace et al (2016) |
| | (SEQ ID NO: 2) | |
| gRNA3 | ACCGAAGUCCCAUAUGCUCC | Janga et al (2018) |
| | (SEQ ID NO: 3) | |
| gRNA4 | CACGUUUUACGAGGGAGAGC | Janga et al (2018) |
| | (SEQ ID NO: 4) | |
| gRNA5 | UCUUGCAGGUGGCACAAACC | Ranganathan Lab |
| | (SEQ ID NO: 5) | |
| gRNA6 | GCCUGUUACUAGCAUUCACA | Ranganathan Lab |
| | (SEQ ID NO: 6) | |
| gRNA7 | GACAUCCCGAGUAUAAAUGC | Ranganathan Lab |
| | (SEQ ID NO: 7) | |
| gRNA8 | GUCUCCAGCUGAUUCGGUCC | Ranganathan Lab |
| | (SEQ ID NO: 8) | |
| gRNA9 | ACGUCGCCCGAACUUUCAGU | Ranganathan Lab |
| | (SEQ ID NO: 9) | |
| gRNA10 | UAAAAGGGUCGCACGUUCGC | Ranganathan Lab |
| | (SEQ ID NO: 10) | |

### Example 6

Allogeneic hematopoietic cell transplantation (allo-HCT) is the most well-established therapeutic option for patients with hematological malignancies who do not respond to conventional treatments. More than 8000 patients receive an allo-HSCT each year in the US alone for the treatment of hematological malignancies and other primary bone marrow disorders. However, the major barrier for the success of allo-HSCT is the high incidence of aGVHD and its associated morbidity and mortality. The pathogenesis of aGVHD involves donor T cell recognition of HLA mismatched host tissues resulting in T cell expansion, migration, and ultimately end-organ damage due to inflammatory cytokine secretion and direct cytotoxic effects. Acute GVHD is clinically characterized by damage to the skin, live, and gastrointestinal (GI) tract.

Prevention of aGVHD is through pharmacological immunosuppression or T cell depletion, but despite the use of standard GVHD prophylaxis regimens, 30-75% of patients who receive allo-HCT eventually develop aGVHD. The standard first-line treatment for GVHD remains high-dose corticosteroids, despite response rates of only 40-60%, resulting in high rates of non-relapse mortality. Among the different treatment strategies for steroid-refractory (SR) GVHD, varying levels of response were seen in clinical trials and many treatments are hindered by increased risk of lethal infections and/or relapse of the primary malignancy. aGVHD remains a major clinical problem emphasizing the need to further determine the pathogenic mechanisms and develop novel therapeutic strategies.

Recent studies have identified critical roles for microRNAs (miRs) in the development and function of the immune system. MiR-155 is required for the normal development of B and T cells and is upregulated upon both B and T cell activation. Given the importance of T cells in the development and progression of aGVHD, as well as the role of miR-155 in the regulation of Th1 responses to antigen presentation, this study investigated the role of miR-155 in aGVHD. miR-155 is upregulated in T cells of mice developing aGVHD following allogeneic transplant. Mice receiving T cells from miR-155^{-/-} donors did not develop aGVHD, but still mounted an appropriate graft-versus-leukemia (GVL) response. Relevant to human disease, dramatic upregulation of miR-155 was also found in clinical specimens from patients with GI aGVHD. In addition, a significant decrease in the expression of a major homing receptor CCR5 was observed on miR-155^{-/-} mouse donor T cells. CCR5 is the principal chemokine receptor that orchestrates the migration of donor T lymphocytes to target organs in recipients. In a phase I/II clinical trial, it was reported that the use of a CCR5 antagonist maraviroc resulted in a lower incidence of aGVHD. Interestingly, targeting miR-155 using antisense oligonucleotides showed very little improvement in survival of mice receiving an allo-transplant. This raises the intriguing prospect that strategies conferring genomic deletion of miR-155 can provide superior protection against aGVHD.

This example investigates gene editing of primary human T cells using the well-established CRISPR-Cas9 system. Earlier approaches to gene editing in T cells involved viral delivery or electroporation mediated transfection, however, these methods had major drawbacks including low efficiency and high toxicity. Recent improvements to CRISPR technology include the use of electroporation of Cas9 ribonucleoprotein (RNPs) that contain a complex of recombinant Cas9 protein with a single guide RNA (sgRNA) which has shown high efficiency (50-90%) in multiple targets such as CXCR4 and CCR5 in primary human T cells. Additionally, the use of high fidelity Cas9 (HiFi Cas9) has been shown to efficiently engineer primary murine and human T cells. HiFi Cas9 significantly reduces off-target effects without affecting on-target efficiency.

This study designed pairs of sgRNAs to target either the promoter or exon 3 of MIR155HG, thus creating genomic deletions that disrupted transcription of the mature miR-155. The CRISPR edited T cells were injected into NSG mice and evaluated for protection against development of GVHD in a xenogeneic model of acute GVHD.

CRISPR/Cas9 Mediated Targeting of MIR155HG Results in Sustained Downregulation of miR-155 Expression in Primary Human T Cells.

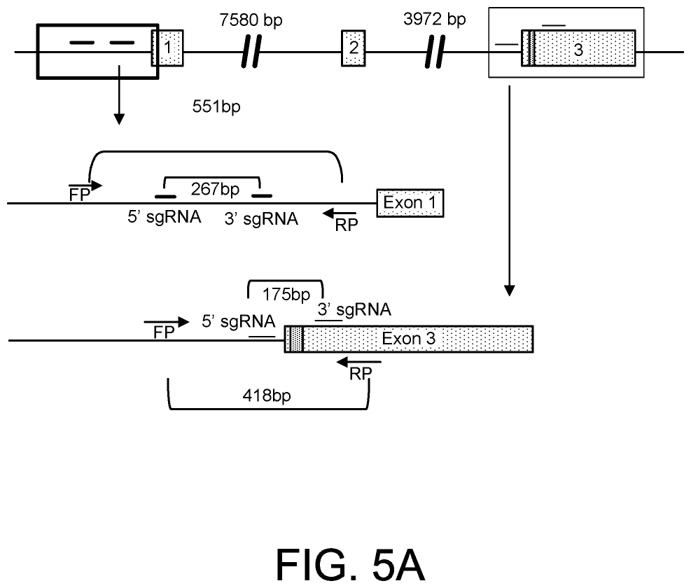

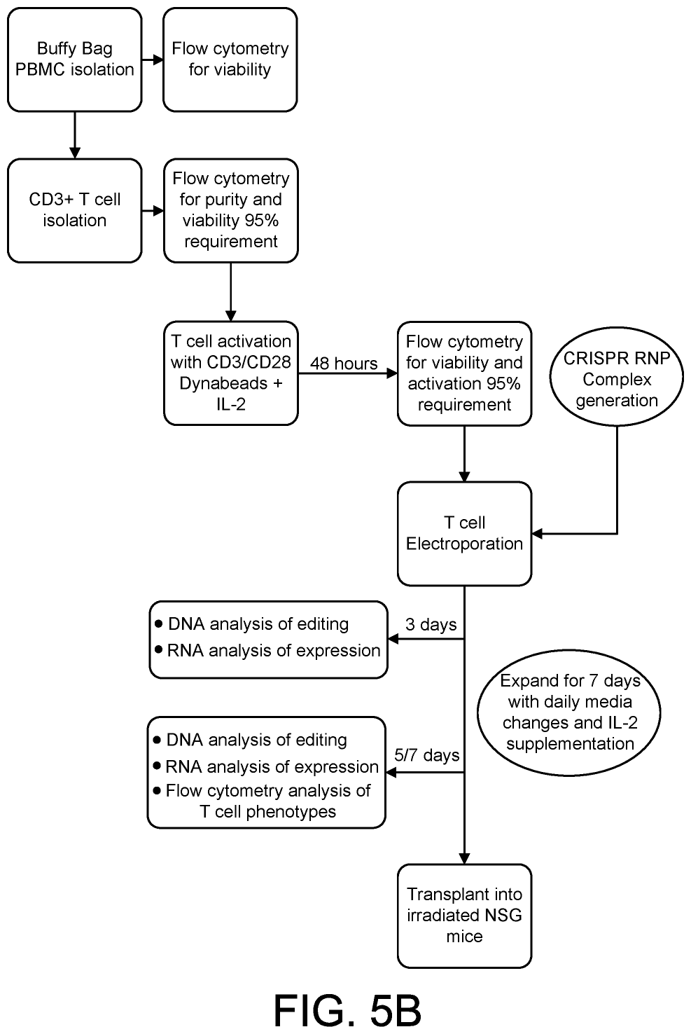

Guide RNAs were designed using Integrated DNA Technologies (IDT) and CHOPCHOP web tools and those predicted to have lowest off-target binding and highest on-target efficiency, while binding to the specific region of interest were selected. Promoter targeting guide RNAs (FIG. 5A) bind flanking the predicted promoter region of MIR155HG and Exon 3 targeting guide RNAs (FIG. 5A) bind flanking the pre-miR-155 sequence, located within exon 3 of MIR155HG. Due to the close proximity of the pre-miR-155 sequence to the 5′ end of exon 3, the upstream guide of the pair was placed within intron 2 of the genomic sequence. Utilizing flanking pairs of guide RNAs creates large deletions of predictable size, removing the region of interest from the genome. Guide RNAs were purchased from IDT as single guide RNAs (sgRNAs) which contain both the crRNA and tracrRNA sequences and chemical modifications for stability. crRNA targeting sequences are listed in Table 2.

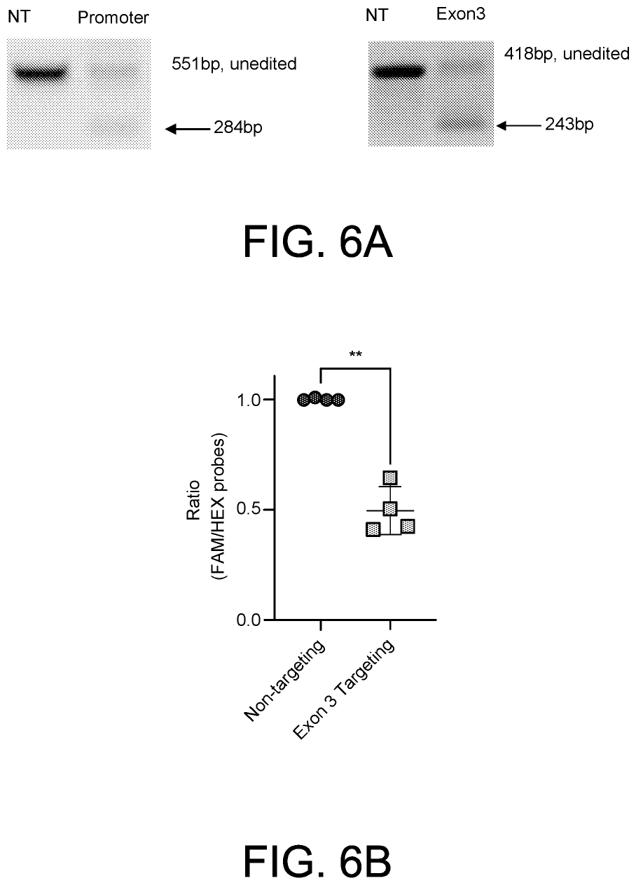

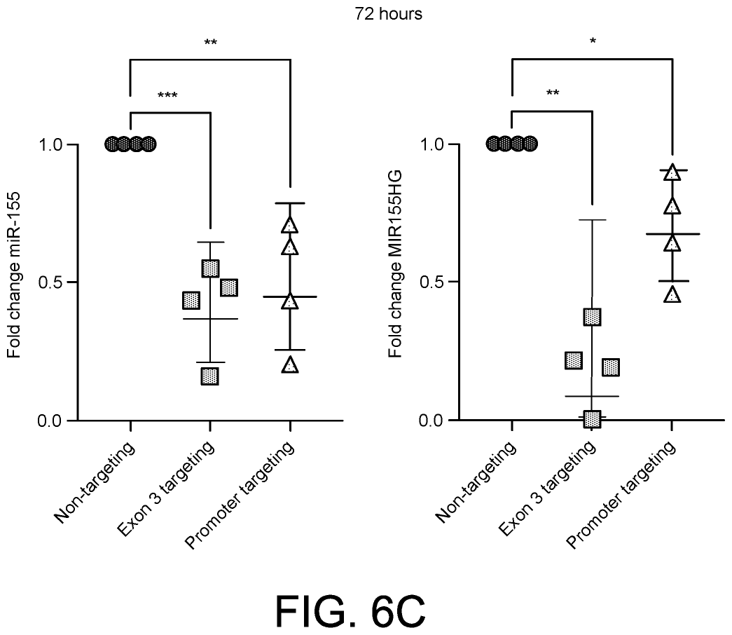

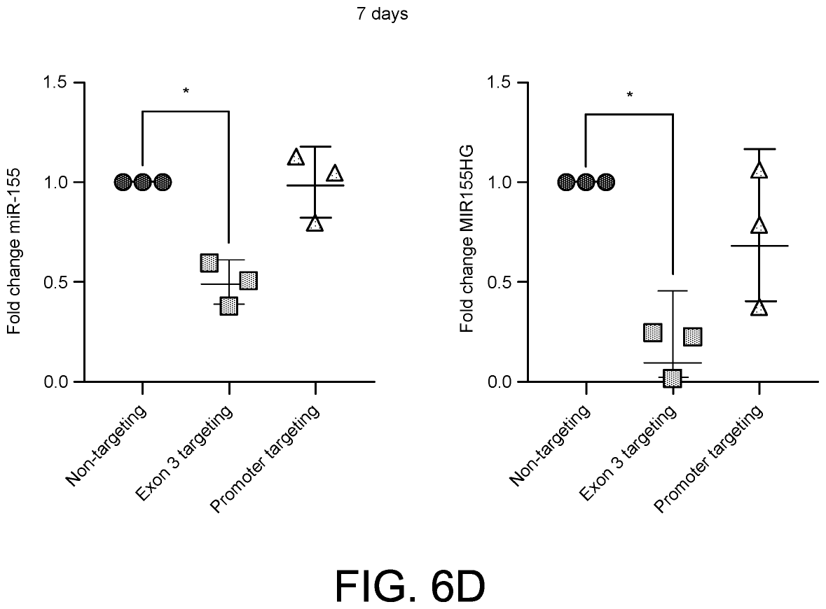

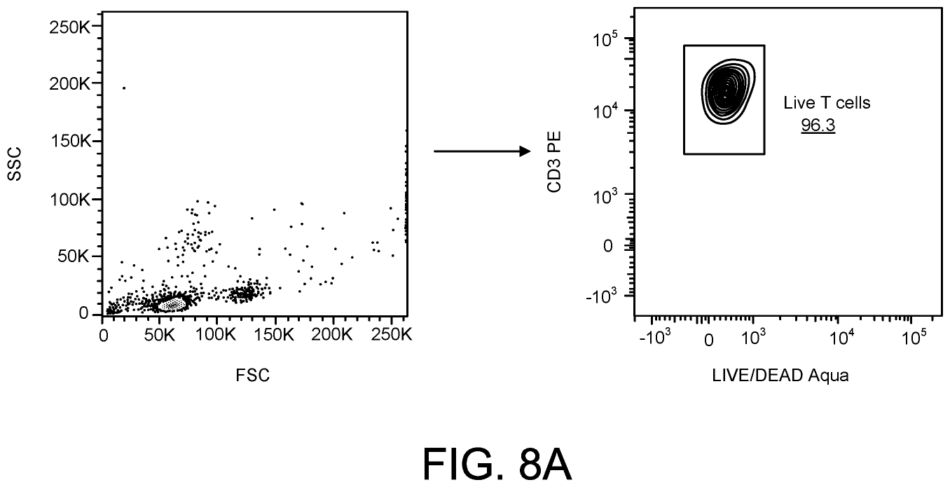

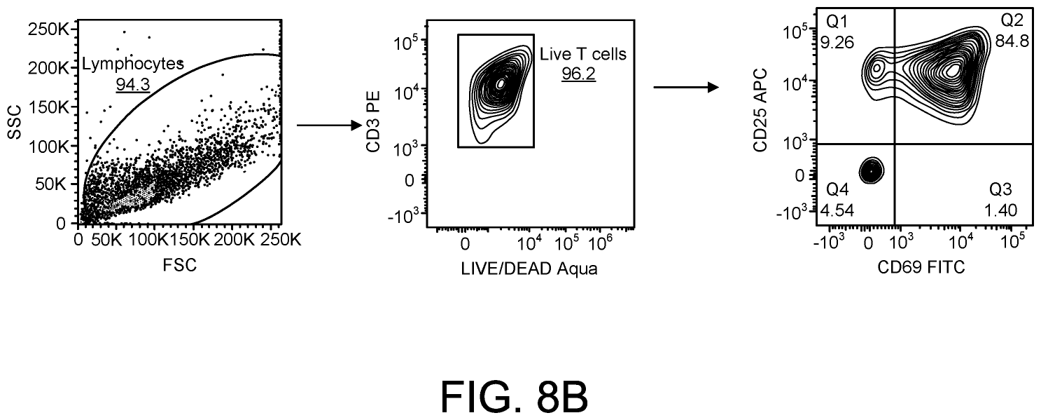

T cells were isolated by negative selection using the Pan T cell isolation kit (Miltenyi Biotec) from fresh PBMCs, confirmed to be greater than 95% pure (CD3+) and viable (PI−) and activated with CD3/CD28 DynaBeads for 48 hours in media supplemented with 20% FBS and IL-2. T cell activation was confirmed by staining for early activation markers CD69 as well as CD25, and only those donors that showed robust activation (>95% CD69 and/or CD25+), were used for transfection (FIG. 8), Activated, viable T cells were electroporated with sgRNAs (non-targeting control (NT), exon 3 targeting, or promoter targeting) and Cas9 enzyme as an RNP complex to ensure efficient editing and minimal off-target effects due to the short-lived nature of the RNP complex. T cells were expanded in media supplemented with IL-2 for 7 days post-transfection to produce sufficient numbers of edited T cells for downstream in vivo experiments. Experimental schema is shown in FIG. 5B. Three days post-transfection, predicted genomic deletions were confirmed using standard genomic PCR with primers flanking the deleted region (Table 2), resulting in a significant size difference in the PCR products produced from non-targeting (NT) control vs. MIR155HG^{Δexon3} (418 bp vs. 243 bp) or MIR155HG^{Δpromoter} (551 bp vs. 284 bp) edited genomic DNA (FIG. 6A).

Quantification of editing efficiency was performed using droplet digital PCR that showed a mean 50% editing using the exon 3 targeting guides. Additionally, quantitative RT-PCR also showed a significant reduction in MTR155HG expression in MIR155HG^{Δexon3} (0.08 vs. 1, p<0.01) and MIR155HG^{Δpromoter} (0.67 vs. 1, p<0.05) compared to NT control. Similarly, miR-155 expression was also significantly downregulated at 72 hours (MIR155HG^{Δexon3} vs. NT control=0.36 vs. 1, p<0.001 MIR155HG^{Δpromoter} vs. NT control=0.44 vs. 1, p<0.001), FIG. 6C. The reduction in expression of MIR155HG (MIR155HG^{Δexon3} vs. NT control=0.09 vs. 1, p<0.05) and miR-155 (MIR155HG^{Δexon3} vs. NT control=1 vs. 0.48, p<0.05) remained significant in MIR155HG^{Δexon3} cells and was sustained through 7 days post-transfection compared to unedited T cells, FIG. 6D.

CRISPR/Cas9 Mediated Targeting of MIR155HG does not Produce Significant Off-Target Effects.

Whole genome sequencing was performed on DNA from unedited, MIR155HG^{Δexon3} edited, and MIR155^{Δpromoter} edited samples from the same human donor. Putative off-target mutations of moderate or high impact are listed in Table 3. Quantitative RT-PCR was performed in unedited, MIR155HG^{Δexon3} edited, and MIR155HG^{Δpromoter} samples from multiple donors to determine if the genomic mutations identified result in changes to transcript expression in edited T cells. No changes in the expression of HDAC7 (MIR155HG^{Δexon3} vs. MIR155HG^{promoter} vs. NT control=1.19 vs. 1.17 vs. 1, ns), KPNA4 (0.86 vs. 0.94 vs. 1, ns), OLFML2A (0.99 vs. 0.91 vs. 1, ns), or POLG2 (0.83 vs. 0.76 vs. 1, ns) were detected between the non-targeting control and either the MIR155HG^{Δexon3} edited or the MIR155HG^{Δpromoter} edited samples across three independent donors. MCC was not expressed to appreciable levels in any of the edited and expanded T cells (Ct undetermined).

CRISPR/Cas9 Edited miR-155 Deficient T Cells Protect from aGVHD in a Xenogeneic Model of Disease.

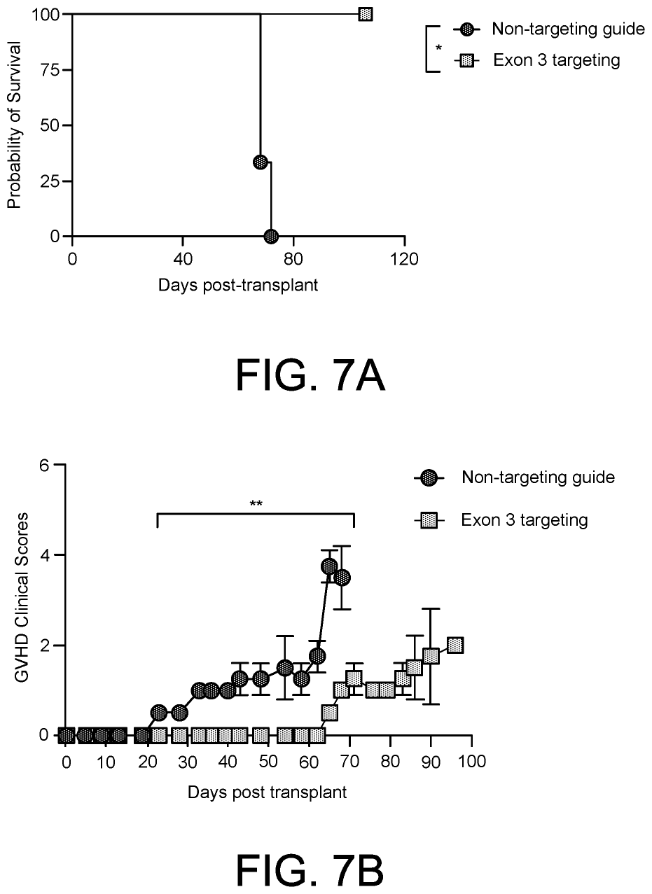

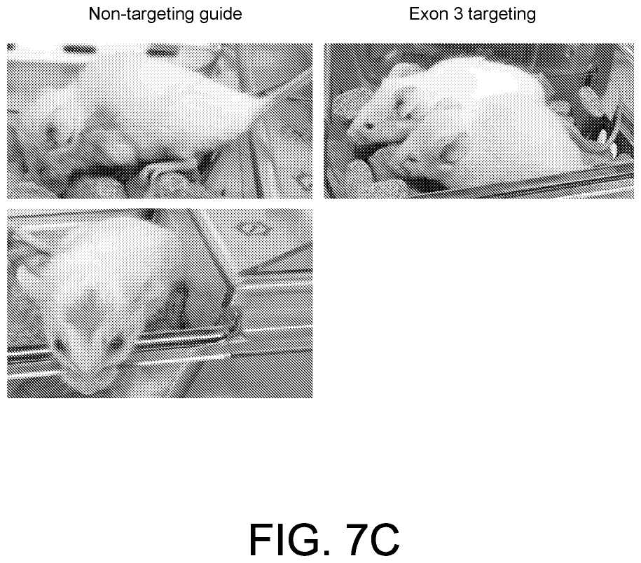

The previously established human PBMC was modified into NSG xenogeneic aGVHD mouse model, instead using in-vitro activated, CRISPR/Cas9 edited and expanded primary human T cells. NSG mice transplanted with MIR155HG^{Δexon3} CRISPR/Cas9 edited T cells survived significantly longer than mice receiving NT control CRISPR/Cas9 transfected T cells (median survival not reached for MIR155HG^{Δexon3} vs. median survival 70 days for NT control, p<0.05, FIG. 7A). Additionally, mice receiving MIR155HG^{Δexon3} edited T cells showed significantly decreased aGVHD clinical scores throughout the duration of the transplant (FIGS. 7B-7C).

### Methods

### Isolation of Human Peripheral Blood T-Lymphocytes

Peripheral blood mononuclear cells (PBMCs) were isolated by Ficoll density gradient centrifugation from buffy coats of healthy human donors obtained from Versiti. T-lymphocytes were isolated from PBMCs using human Pan T cell isolation kit (Miltenyi Biotec), Isolated T cells were seeded at a density of 1*10\hat{ }6 cells/ml in a 25 cm² culture flask in RPMI 1640 medium supplemented with 20% FBS and 1% Penicillin-Streptomycin (Pen-Strep) and 1% Glutamine (Gibco).

### Activation of Human Peripheral Blood T-Lymphocytes

T cells isolated from fresh PBMCs were activated and expanded using Dynabeads Human T-Activator CD3/CD28 (Thermo Fisher Scientific) at 0.5×10⁶ cells per mL of media (RPMI+20% FBS+1% PenStrep+1% Glutamine)+30 U/mL recombinant human IL-2 (R&D Systems). Cells were incubated at 37° C./5% CO₂ in a humidified incubator for 48 hours.

### Flow Cytometry Analysis

Cells were stained at various timepoints to assess purity, viability, activation, and phenotype. Approximately 0.5-1×10⁶ T cells were stained with surface antibodies and viability dyes following manufacturer's protocols. Analysis was performed on the FACS LSRFortessa flow cytometer (Becton Dickinson) or the Aurora Northern Lights (Cytek) depending on the size of the panel. Data analysis was performed using FlowJo (Tree Star). Representative gating strategies can be found in the FIG. 8.

### CRISPR-Cas9 Gene Editing of MIR155HG

CRISPR mutagenesis was performed on activated T cells using ribonucleoprotein (RNP) complex. Briefly, one or two single guide RNAs (Alt-R® CRISPR-Cas9 sgRNA, IDT) were incubated with Alt-R® S.p. Cas9 Nuclease V3 (IDT 1081059) in Buffer T (Thermo Fisher Scientific) at room temperature for 10 minutes in approximately 1.4:1 ratio to form the RNP complex. RNP complex was mixed with activated T cells (1×10⁶) and electroporation performed using the Neon Transfection System (Thermo Fisher Scientific). Electroporation conditions were 1600 V, 10 milliseconds, 3 pulses, using buffer T. Electroporated cells were seeded at a density of 0.5×10⁶ cells/ml in a 6 well plate in RPMI-1640 medium supplemented with 20% FBS and 1% L-glutamine (antibiotic-free). The flask was incubated at 37° C./5% CO₂ in a humidified incubator. Fresh antibiotic-containing media (as for activation of T cells) and IL-2 were added based on the replication of cells to maintain cell density.

### DNA Analysis for Editing

DNA was extracted and purified (DNeasy Blood & Tissue Kit, 69504 Qiagen) from human T cells at 3, 5, and 7 days post-transfection. Standard genomic PCR amplification was performed using DreamTaq 2×Master Mix (ThermoFisher) and primers listed in Table 2. DNA products were separated on a 2% agarose gel. Gel images were obtained. CRISPR-Cas9 on-target efficiency was assessed using QX200 droplet digital PCR (ddPCR) System. A drop-off FAM probe (custom design, Biorad) was designed that would bind between two sgRNAs in the unedited allele. A reference Hex probe (custom design, Biorad) binds upstream of the first sgRNA in both the unedited and edited alleles. The frequency of gene editing in total human T cells were calculated by the ratio of droplets containing FAM signal to droplets containing HEX signal.

### Quantitative RT-PCR

Total RNA was isolated from human T cells by Trizol extraction following manufacturer's instructions. MIR155HG and miR-155 cDNA synthesis was carried out using RevertAid RI Reverse Transcription Kit (ThermoFisher Scientific) and TaqMan™ MicroRNA Reverse Transcription Kit (ThermoFisher Scientific) respectively. MIR155HG and miR-155 expression levels were detected using TaqMan human gene expression (ThermoFisher Scientific, Assay ID Hs01374570_ml) and miRNA (Assay ID 002623) assays. Normalization was performed using Hprt and RNU44 expression levels for MIR155HG and miR-155 respectively.

### Whole Genome Sequencing for CRISPR-Cas9 Off-Target Effects

DNA samples from untransfected and edited T cells were sequenced and analyzed by the genomics core facility at Nationwide Children's Hospital (NCH). CRISPR-Cas9 off-target effects were assessed by Whole-Genome Sequencing (WGS) and CHURCHILL downstream analysis. Churchill enables for the computationally efficient examination of a high-depth whole genome sample. Churchill entirely automates the laborious and computationally complex process of aligning, post-alignment processing, and genotyping, resulting in a variation list appropriate for analysis. Reads from Churchill analysis were aligned using BWA MEM (v0.7.15) to the MIR155HG reference genome. Variants were called using the Mutect2 tool of the Genome Analysis Tool kit (GATK v4.0.5.1, Broad Institute) and annotated using SnpEff (v4.3). Single nucleotide polymorphisms and insertion-deletion mutations (indels) that are exclusive to the edited cells were detected when compared with unedited T cells from the same donor. Off-target mutations were validated using genomic PCR and Sanger sequencing.

Xenogeneic aGVHD Mouse Models

Mice were transplanted under standard protocols approved by the University committee on Use and Care of Laboratory Animals at OSU. Only age- and sex-matched NOD-scid IL2Rgamma^{null} (NSG) mice were used. Briefly, 8 to 10-week recipient NSG mice were irradiated with 50 cGy the day prior to transplantation. Edited and expanded T cells (˜5-10×10⁶) were administered on the day of the transplant via tail-vein injection.

Clinical and Histological Assessment of aGVHD

Recipient NSG mice were weighed 2-3 times per week and monitored daily for clinical signs of aGVHD and survival. GVHD scores were performed. This scoring system incorporates parameters of weight loss, activity, posture (hunching), fur texture, and skin integrity. Individual mice were ear tagged and graded (on a scale of 0 to 8) 2-3 times per week. Mice that received an acute GVHD score of ≥7 were euthanized and their tissues harvested. Detailed histopathology analysis of liver, spleen, gut, skin, ocular, and bone tissues was also performed. A previously reported scoring system with a range of 0 (no signs of GVHD) to 4 (maximal GVHD damage) was used to score histopathological samples. An experienced pathologist read all the samples in a blind study.

TABLE 2
Guide RNA sequences and
primer sequences for genomic PCR

| Primer Name | Sequence (5′-3′) |
|---|---|
| MIR155HG promoter: | GACAUCCCGAGUAUAAAUGCGGG |
| 5′ gRNA | (SEQ ID NO: 11) |
| MIR155HG promoter: | ACGUCGCCCGAACUUUCAGUCGG |
| 3′ gRNA | (SEQ ID NO: 12) |
| MIR155HG exon 3: | UCUUGCAGGUGGCACAAACCAGG |
| 5′ gRNA | (SEQ ID NO: 13) |
| MIR155HG exon 3: | AUGGAACAAAUUGCUGCCGUGGG |
| 3′ gRNA | (SEQ ID NO: 14) |
| MIR155HG promoter | AAATCAAATGGCTGCTCCAG |
| fwd primer | (SEQ ID NO: 15) |
| MIR155HG promoter | GGGCTCCAACCTTTGTTCTT |
| rev primer | (SEQ ID NO: 16) |
| MIR155HG exon 3: | TGAGCTCCTTCCTTTCAACA |
| fwd primer | (SEQ ID NO: 17) |
| MIR155HG exon 3: | TGAACATCCCAGTGACCAGA |
| rev primer | (SEQ ID NO: 18) |
| HRNR fwd | ATCTAGGGGTGAACGACACG |
| | (SEQ ID NO: 19) |
| HRNR rev | GAATACCCATCTTGCCCTGA |
| | (SEQ ID NO: 20) |
| HRNR fwd | CTCAGGGCACTTCTGTAGCC |
| | (SEQ ID NO: 21) |
| HRNR rev | TGGAAGGAACCCCATAACAA |
| | (SEQ ID NO: 22) |
| HDAC7 fwd | GTGTGTGGACAGGTGTGAGG |
| | (SEQ ID NO: 23) |
| HDAC7 rev | GCCTCGGAGACACTGAAAAG |
| | (SEQ ID NO: 24) |
| IGFN1 fwd | TAGGGGTGGCTTAGGTTCTG |
| | (SEQ ID NO: 25) |
| IGFN1 rev | AGGAACCCCCAAGTCC |
| | (SEQ ID NO: 26) |
| KPNA4 fwd | CAGGATTGCCTGAGCTTTGT |
| | (SEQ ID NO: 27) |
| KPNA4 rev | GCTGTTGAGGACAAGAAATGC |
| | (SEQ ID NO: 28) |
| MCC fwd | GAGCCCAGCTCTGAAAAGTG |
| | (SEQ ID NO: 29) |
| MCC rev | TCAGCCTAGGGAAGAGACCA |
| | (SEQ ID NO: 30) |
| OLFML2A fwd | TGGGCTCCTGACTCTCAGTT |
| | (SEQ ID NO: 31) |
| OLFML2A rev | GCTGGAGACTTCTGGACCTG |
| | (SEQ ID NO: 32) |
| POLG2 fwd | GCTTTTTCCCCTTTCCTGTC |
| | (SEQ ID NO: 33) |
| POLG2 rev | CATATTGGCCAGGATGGTCT |
| | (SEQ ID NO: 34) |

TABLE 3
Off-target mutations from CRISPR-Cas9 editing identified by WGS

| | | | | | Samples |
|---|---|---|---|---|---|
| Chr | Start | Gene | MANE Transcript HGVS | Impact | Containing |
| 1 | 152213347 | HRNR | NM_001009931.3(HRNR): | Moderate | MIR155HG^{Δexon3} |
| | | | c.8282G > A: p.Arg2761Gln | | MIR155HG^{Δpromoter} |
| 12 | 47791924 | HDAC7 | NM_015401.5(HDAC7): | High | MIR155HG^{Δexon3} |
| | | | c.17551758delCCGC: p.Arg586fs | | MIR1SSHG^{Δpromoter} |
| 1 | 201209539 | IGFN1 | NM_001164586.2(IGFN1): | Moderate | MIR155HG^{Δexon3} |
| | | | c.4646C > G: p.Thr1549Arg | | MIR155HG^{Δpromoter} |
| 1 | 152213341 | HRNR | NM_001009931.3(HRNR): | High | MIR155HG^{Δexon3} |
| | | | c.8286_8287delTG: p.His2762fs | | MIR155HG^{Δpromoter} |
| 3 | 160535883 | KPNA4 | NM_002268.5(KPNA4): | Moderate | MIR155HG^{Δexon3} |
| | | | c.128_129delAAinsTT: p.Glu43Val | | MIR155HG^{Δpromoter} |
| 3 | 160535893 | KPNA4 | NM_002268.5(KPNA4): | Moderate | MIR155HG^{Δexon3} |
| | | | c.118_119delAAinsTT: p.Lys40Leu | | MIR155HG^{Δpromoter} |
| 5 | 113294355 | MCC | NM_001085377.2 > NM_001085377.2(MCC): | Moderate | MIR155HG^{Δexon3} |
| | | | c.627 + 46164A > C | | MIR155HG^{Δpromoter} |
| 9 | 124804258 | OLFML2A | NM_182487.4(OLFML2A): | High | MIR155HG^{Δexon3} |
| | | | c.1086delC: p.Thr363fs | | |
| 17 | 64482917 | POLG2 | NM_007215.4(POLG2): | High | MIR155HG^{Δpromoter} |
| | | | c.1191 + 2T > A | | |

TABLE 4
MIR155HG is encoded on the + strand of human chromosome 21 from
25,562,048 to 25,575,168 (SEQ ID NO: 45 is the sequence) (Gene:
ENSG00000234883.7, Transcript: ENST00000659862.2).
Gene Structure:

| | Start | End | Length |
|---|---|---|---|
| Promoter | unannotated | | |
| Exon 1 | 25,562,048 | 25,562,257 | 210 |
| Intron 1 | 25,562,258 | 25,569,836 | 7,579 |
| Exon 2 | 25,569,837 | 25,569,921 | 85 |
| Intron 2 | 25,569,922 | 25,573,892 | 3,971 |
| Exon 3 | 25,573,893 | 25,575,168 | 1,276 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
gRNA1
SEQ ID NO: 1
GUUAAUGCUAAUCGUGAUAG,
gRNA2
SEQ ID NO: 2
GCUAAUAUGUAGGAGUCAGU,
gRNA3
SEQ ID NO: 3
ACCGAAGUCCCAUAUGCUCC,
gRNA4
SEQ ID NO: 4
CACGUUUUACGAGGGAGAGC,
gRNA5
SEQ ID NO: 5
UCUUGCAGGUGGCACAAACC,
gRNA6
SEQ ID NO: 6
GCCUGUUACUAGCAUUCACA,
gRNA
SEQ ID NO: 7
GACAUCCCGAGUAUAAAUGC,
gRNA8
SEQ ID NO: 8
GUCUCCAGCUGAUUCGGUCC,
gRNA9
SEQ ID NO: 9
ACGUCGCCCGAACUUUCAGU,
gRNA10
SEQ ID NO: 10
UAAAAGGGUCGCACGUUCGC,
(SEQ ID NO: 11)
GACAUCCCGAGUAUAAAUGCGGG
(SEQ ID NO: 12)
ACGUCGCCCGAACUUUCAGUCGG
(SEQ ID NO: 13)
UCUUGCAGGUGGCACAAACCAGG
(SEQ ID NO: 14)
AUGGAACAAAUUGCUGCCGUGGG
(SEQ ID NO: 15)
AAATCAAATGGCTGCTCCAG
(SEQ ID NO: 16)
GGGCTCCAACCTTTGTTCTT
(SEQ ID NO: 17)
TGAGCTCCTTCCTTTCAACA
(SEQ ID NO: 18)
TGAACATCCCAGTGACCAGA
(SEQ ID NO: 19)
ATCTAGGGGTGAACGACACG
(SEQ ID NO: 20)
GAATACCCATCTTGCCCTGA
(SEQ ID NO: 21)
CTCAGGGCACTTCTGTAGCC
(SEQ ID NO: 22)
TGGAAGGAACCCCATAACAA
(SEQ ID NO: 23)
GTGTGTGGACAGGTGTGAGG
(SEQ ID NO: 24)
GCCTCGGAGACACTGAAAAG
(SEQ ID NO: 25)
TAGGGGTGGCTTAGGTTCTG
(SEQ ID NO: 26)
AGGAACCCCCAAGTCC
(SEQ ID NO: 27)
CAGGATTGCCTGAGCTTTGT
(SEQ ID NO: 28)
GCTGTTGAGGACAAGAAATGC
(SEQ ID NO: 29)
GAGCCCAGCTCTGAAAAGTG
(SEQ ID NO: 30)
TCAGCCTAGGGAAGAGACCA
(SEQ ID NO: 31)
TGGGCTCCTGACTCTCAGTT
(SEQ ID NO: 32)
GCTGGAGACTTCTGGACCTG
(SEQ ID NO: 33)
GCTTTTTCCCCTTTCCTGTC
(SEQ ID NO: 34)
CATATTGGCCAGGATGGTCT
5′ promoter targeting sgRNA binding site (for PrA)
(SEQ ID NO: 35)
GACATCCCGAGTATAAATGCGGG
5′ promoter targeting sgRNA binding site (for PrB)
(SEQ ID NO: 36)
GTCTCCAGCTGATTCGGTCCAGG
3′ promoter targeting sgRNA binding site (for both PrA and PrB)
(SEQ ID NO: 37)
CCGACTGAAAGTTCGGGCGACGT
5′ Exon 3 targeting sgRNA binding site, crosses the intron 2-exon 3 boundary
(SEQ ID NO: 38)
TcttgcagGTGGCACAAACCAGG
3′ Exon 3 targeting sgRNA binding site
(SEQ ID NO: 39)
ATGGAACAAATTGCTGCCGTGGG
Putative upstream exon, only annotated in transcript variant ENST00000456917.2
(SEQ ID NO: 40)
TACACACACGCAATGACCCACGAGAAAGGGAAAGGGGAAAACACCAACTACCC
GGGCGCTGGGCTTTTTCGACTTTTCCTTTAAAAAGAAAAAAGTTTTTCAAGCT
EXON 1 of miR155 host gene
(SEQ ID NO: 41)
GCGCGGGCTTCCTGTGCGCGGCCGAGCCCGGGCCCAGCGCCGCCTGCAGCCTCG
GGAAGGGAGCGGATAGCGGAGCCCCGAGCCGCCCGCAGAGCAAGCGCGGGGAA
CCAAGGAGACGCTCCTGGCACTGCAG
EXON 2 of miR155 host gene
(SEQ ID NO: 42)
ATAACTTGTCTGCATTTCAAGAACAACCTACCAGAGACCTTACCTGTCACCTTGG
CTCTCCCACCCAATGGAGATGGCTCTAATG
EXON 3 of miR155 host gene
(SEQ ID NO: 43)
GTGGCACAAACCAGGAAGGGGAAATCTGTGGTTTAAATTCTTTATGCCTCATCCT
CTGAGTGCTGAAGGCTTGCTGTAGGCTGTATGCTGTTAATGCTAATCGTGATAGG
GGTTTTTGCCTCCAACTGACTCCTACATATTAGCATTAACAGTGTATGATGCCTGT
TACTAGCATTCACATGGAACAAATTGCTGCCGTGGGAGGATGACAAAGAAGCAT
GAGTCACCCTGCTGGATAAACTTAGACTTCAGGCTTTATCATTTTTCAATCTGTTA
ATCATAATCTGGTCACTGGGATGTTCAACCTTAAACTAAGTTTTGAAAGTAAGGT
TATTTAAAAGATTTATCAGTAGTATCCTAAATGCAAACATTTTCATTTAAATGTCA
AGCCCATGTTTGTTTTTATCATTAACAGAAAATATATTCATGTCATTCTTAATTGC
AGGTTTTGGCTTGTTCATTATAATGTTCATAAACACCTTTGATTCAACTGTTAGAA
ATGTGGGCTAAACACAAATTTCTATAATATTTTTGTAGTTAAAAATTAGAAGGAC
TACTAACCTCCAGTTATATCATGGATTGTCTGGCAACGTTTTTTAAAAGATTTAGA
AACTGGTACTTTCCCCCAGGTAACGATTTTCTGTTCAGGCAACTTCAGTTTAAAA
TTAATACTTTTATTTGACTCTTAAAGGGAAACTGAAAGGCTATGAAGCTGAATTT
TTTTAATGAAATATTTTTAACAGTTAGCAGGGTAAATAACATCTGACAGCTAATG
AGATATTTTTTCCATACAAGATAAAAAGATTTAATCAAAAAATTTCATATTTGAA
ATGAAGTCCCAAATCTAGGTTCAAGTTCAATAGCTTAGCCACATAATACGGTTGT
GCGAGCAGAGAATCTACCTTTCCACTTCTAAGCCTGTTTCTTCCTCCATATGGGG
ATAATACTTTACAAGGTTGTTGTGAGGCTTAGATGAGATAGAGAATTATTCCATA
AGATAATCAAGTGCTACATTAATGTTATAGTTAGATTAATCCAAGAACTAGTCAC
CCTACTTTATTAGAGAAGAGAAAAGCTAATGATTTGATTTGCAGAATATTTAAGG
TTTGGATTTCTATGCAGTTTTTCTAAATAACCATCACTTACAAATATGTAACCAAA
CGTAATTGTTAGTATATTTAATGTAAACTTGTTTTAACAACTCTTCTCAACATTTT
GTCCAGGTTATTCACTGTAACCAAATAAATCTCATGAGTCTTTAGTTGATTTAAA
ATAA
Host gene encoding the Pre-miR-155 sequence
(SEQ ID NO: 44)
TTAATGCTAATCGTGATAGGGGTTTTTGCCTCCAACTGACTCCTACATATTAGCAT
TAACAG
>MIR155HG with PROMOTER
MIR155HG is encoded on the + strand of human chromosome 21 from 25,562,048 to
25,575,168 (Gene: ENSG00000234883.7, Transcript: ENST00000659862.2). Promoter and
introns are in lowercase, exons are in uppercase
(SEQ ID NO: 45)
gatcaagatgtcagcaggactggttcctcctgagagctgtgagggaaggatctgtttcaagcccctctccttggctggtagatggccat
cttttcccaatgtttcttcacactgtcttccttctatatgtgtttctttctctacacatcatgttctttttctaagaacaccagtcatattggattagg
agccaaccctattttaataagacctcatcttgactaattacatccacaaaggccttgtttccacataaggttatattctgaagtactggttggt
ctacagccataccaccccacacacacccgatctcatctgaagtactgggtgttaggacatcaatatatgaatttgggaggtgggggga
gggggacacaattcagcccacaacaacatccatggggtacttgtcccggcaggtggccagctcttactttctcaagctatccatttgga
gcaggtccttttttttttttctttttttttttttttgagacagtctcgctcgcattccaggctggaatgcagtggcgcgatcttggctcaccgcaaa
ctccgcctcccgcattcatgccattctcctgcctcagcctccagagtagctgggactacaggtgcccgccatcatgcccggctaatttttt
tttcttttttgtatttttagtagattcagggtttcaccattctagccaggatggtctcaatctcctgacctcatgatccagtcacctcagcctccc
aaagtgctgggattacaggatgagccaccgcgcctggcctggagcaggtactttttatccatcaccttatttaatcatcatatccctcttag
tctgctagggttgccataagaaaataccatagactggatggctgatacaacaacaatttattttctcacattctggaggctagaaagttcca
gatcaaggtcctgcagggttcggtttctggtgaggactctcttcctggcttacaggaaaccaccatcccattgtgtgctcacatgacctct
gctttgactgatttctgagagaaagggaattctggtgtctcttcctcttctcataagagcactagctctatgggattagtgatccacccttttg
acctcatttaacctttatcacctcctcacaggccctatctccaaatggggataacattggggatcagggcttcaacatataaagttgaaga
gaacatgattcagtctatagtaatccctgatgggagacatcattattgtcatttacaggaagctgagacataaaaagtttaacgttcagatt
ccacagctagaattagtgtaaaatgcaggtgtatttgactcctaaaacccatttatccatccactattctgggtgtctcaaactgggaccta
aaatgcggaggtttgcacatgacctgaaattacttccaccactgggagaagggaggattgagcaatttcctgtttctattaaagtgcattat
acaaacttgtcattctggggatgaaaggtcaccctagaattgcctatgggcaatttcttatagttcaacctagaatgagaaatgggaaatt
cagaaaggcattgtaggcatctgtaaccagcagagggacgtgccccacctgggtggggaccatgcatccttgccacatgccccactg
cacaacttccccagctcctcaacgtcacatggatctggaaagcagggagactggactacggagccagccctccagggttggaactga
gtttgaatctcagcttgactacttactaggatctaaggtaacatgtttaatctctctgtgctcagtttcctcatctggaaatagagattataatg
ctcctccctcatatgatcatagtgaggactgaaagagttaatgcatataaggagtgttaggacaggatctggcacatggtaaatgctttat
aagtgttagttgttatcatcatgattattcttgtttgaattataagagaaaaagcatgtatcttaagaatagagggttttaaaatgctcccaagt
tccttaaccaacctgagccatctgtaaattaagtactatgggatttccagctctgacatgtattctgacatgtaactggcatcagtttaataca
aactattctaaaatgtcttgtgcccttgacagggaggtccaagtactggatacttgcaatgcaatccagcagtggccccgtctttcttacaa
aaaggccccagtcacatgttgatgaggctagatctattcctgtcctccttctcttccccatatttccttattcttcttcaagtctaaacgtttatt
gaactgagacccatgaatgagttactcgactaggcttgtaggataaacttgccaggctttagatctatttgttcctttccatcccccaaaatc
aaatggctgctccaggaaaatgttccccttgtggcagggtccgggagaaaagagagaagcgacaaaaccaaaaattaaaacgaccg
aagtcccatatgctccaggaatatgtcctggagatgggagtggagggcagggggagaatgttgttgaggtcaaaatttttgaagttttaa
gtcctatatcttgacatcccgagtataaatgcgggtaccagacacagtacaaacgttctcaaagcccagttacgtattccaaaccaaacg
cgggctcttgaagggtgatgaggtagggatgaaatccaggatcgcctgaagaccatttcttcctctcttagggacctgctggtctccag
ctgattcggtccaggaggaaaaacctcccacttgctcctctcgggctccctgcaaggagagagtagagacactcctgccacccagttg
caagaagtcgccacttccccctccagccgactgaaagttcgggcgacgtctgggccgtcatttgaaggcgtttccttttctttaagaaca
aaggttggagcccaagccttgcggcgcggtgcaggaaagtacacggcgtgtgttgagagaaaaaaaaTACACACACGC
AATGACCCACGAGAAAGGGAAAGGGGAAAACACCAACTACCCGGGCGCTGGGC
TTTTTCGACTTTTCCTTTAAAAAGAAAAAAGTTTTTCAAGCTgtaggttccaagaacaggcag
gaggggggagaagggggggggggttgcagaaaaggcgcctggtcggttatgagtcacaagtgagttataaaagggtcgcacgttc
gcagGCGCGGGCTTCCTGTGCGCGGCCGAGCCCGGGCCCAGCGCCGCCTGCAGCCT
CGGGAAGGGAGCGGATAGCGGAGCCCCGAGCCGCCCGCAGAGCAAGCGCGGGG
AACCAAGGAGACGCTCCTGGCACTGCAGgtacgccgacttcagtctcgcgctcccgcccgcctttcctctct
tgaacgtggcagggacgccgggggacttcggtgcgagggtcaccgccgggttaactggcgaggcaaggcgggggcagcgcgca
cgtggccgtggagcccggcctggtcccgcgcgcgcctgcgggtgccccctggggactcagtggtgtcgcctcgcccgggaccag
agattgcgctggatggattcccgcgggcagaggcagggggaaggaggggtgttcgaaacctaatacttgagcttctttgcaaagtttc
cttggatggttggggacgtacctgtataatggccctggaccagcttccctgttggagtggccagagaagtgtgtaaaacacactagag
gggcagggtggaaaaagagactgccttcaaaacttgtatcttttcgatttcattttgaaaaataactacaaatctattttaattttacaaagtta
gactcatagcattttagatatcaatgtcttcatttaacagaagtgaagatggagcaaacgctcaatcagcgtctgtatttattcgctcctgttg
tgccagggtgcgtttttgccgagcggttgcctttctttactcacaaaacccccttgatgtctgtcctccacgttttacgagggagagccgg
atcttttgaagtttgtatcatctaaagcaggtatattgggatgactatggatagaatttaacctgaaaacactgaagttgacagctgacaaa
gatataagaatccaaagtatgttaaaaattaaggagctgaggcccacagaagtgaagttacttttccagcatcacacagcagatctagg
accctgtgtgatttgaaaaggccaaggtatctaatgatgctcataatttagtttacattttgtgtcaaatagatgaggtcaagtggacattatg
tgtatattccaaagccaaaattactgcttgtaatatccattcatctctccgaagacgtagtgcctttttatatggctttaacctaatgtataatc
gaagtgaattagggcagccagacttttaatcacaggactgatcttttaaaaacgggtatatgtaaagttaattgcttatctatgtgaacagta
atatacactataaaaagttttcaaggcaacttcctgcctgtggtttaatttgatagtcacaatgataggaaattggcagggttaggtggtggt
atctacattttgcccatgaggacttgtggctcagagaatttgagttttttctagtaaggtcactaggtccctagtgcacagcctgcttttaggt
acggatgtgggatgggggtgtcggcagaccacagggttttgactctcagtttgggctcatttccatgactccttgctgcctccctgatttct
gtcaaatgaccaaagcacatttagtaattacttctgctgcaaggactactccttattaggctgtgataagtaagtttcctcacatagtgggtc
agctcactctggccacaggacccagcttcctaaccacacacattaagaaagagaaaaattagcactgtctgagacctacaaccacttca
agggagaacagtggtgtttgcacaaatgtctacttttgttttaacctagtcatgtgtaaaaagtgtaattcactggtctttaccaaaaaaaaa
aaaaaaaaaacaacaacaaaaaaaaaacaggcatcatagtctgcctgtgctggacgctatgctagacactggagagacaaaggtga
gcaggataggcacaaccctacccttacaaacccagaaagttagaggaaaggccatcaggacattgctgtgcaatttgatatcatttcaa
ttttggtaacatagatatttgaactaattagaatgtgctacaaattttgccattctttactttccatatgccaacatggaaaatttgtaaccctcat
ctttaaatacctcatcttaccagtcctctataaatcaaaaaatctaccccaaaaagccctaggttttaacttttgaaaagtttttttttatcagctt
ttcaggtcatgactaagatctgccattcaatttaaaggaattctttcattttagagtaatagttactaaaatcctttattcctacctccttgagaat
ctaatggaaattttatatgctcttcccagaaaatttttgttaatgtgtatcctcacacaccaatttgcattcgatgttagcaggctagagacttc
cttgaatctcagccatgaccctcttcgggggtccactttcataatcagtattctaaacctagtaggcataaatagcttgaatagaggaaagt
ggtatgtagactcttgcccttcacttggactgggcacctctatcacttggactgggcaccagctagtgcccttaactagctggtgtctccat
ttttgatagtcattggctattgcttagctgaagctattgattttaacagccttattgttttgatatcaggctgccactctgcagaagtctaatact
ggggaaggagcaccggcctgagaaccatggtttgtttctaggaccaactctgcagatttctgtgcctcattttgctcatctctaagatggg
tggttgatgggtggcagggactgaaccatttctaaaatcccttacactgatgaaattctgtgattccagtgtaactaccaagctcactttcat
atacctcctgagacacagctggtcttagtgaatttatatcctctatgtccaaagtaagtcaaagtaagtaccactataattcactatagaatg
tatttttgtctttcactgatcttactggactcaaaactgttacatgtttccttaaattagatagaataaaaaaattgtgaaacactacttttcaagg
aaaaatattacaattttaaaagcctctttttatcccacctctcagtagctacaactttcatatacgagtttcacatatatatgaaaaaaattttctt
atctcagactcttaaggaaatgcaatgcttttaatatgatcactgctgtgtaattctcatctgccctgaaacacttgttctgtccatcaatcaat
gtatttgggtttacaatatgcagagcctcaagcaaaaccagtgcaaaagcacatctatcctcaaataggcacatcatatgtctgagcagtt
cagcatcctcactcacaagtccaaggctctcagcaatgtggaagaagatgtgattcctaaaattaccacccttacacacagtgacacact
caccagaaagtgaggcttaacatagcttaagtggctgtagtttaaaacagattgtgaatatctgtgctaattctgagaaacaattagcaga
catattaaccaattagcacagatcactaatatctgtgctaattgttaggacagattagcacagatattgctaccttgtgtgggtagacaggg
ttaaggcactggcagaaggaaagattagtgaggaatatcaaatagctgatttaatgaaattgtgaggccgatgtttggataaggtgaaac
agtgccctacaatgacatttatgccagttctccataaacaagagtgttctacacaaattataagttcctggctgtcttaacaaaatgctatgg
tcctttatatgctcacacaaataatggttccaagcatgggaaacaggtgccagattgcccaggtaggcatcccagcagcatgctccttag
ccactgattaaccttgcgcaagttatttcacctgcctgtgcctcaaggcccctgtgtttcaatggggagggtgtgaatactcattcctaagg
ttgtgaggaggaataactgagttagtatacgtcaaacacttaggtgcttggcacagagggagcctgtgtaagtgtttgctattattttgtttc
actagatgatcttgtgtattataagaaatcaatttggaaggtgagatacaaatgcagtctgagaaagtaaagacgttttaaagacattttact
gtgtcatgaattttcagagaccacactgacatggtaaaaagttgtttggtttttagaatttttgtttcagaatgcatatccttctccttaaccaaa
agaaaaactactaaaatacttctgaaaattatttccaagaaaacattttggtgtaccttttagcagttactttatgacttaaagattaggatgaa
caaaataattattttaacttggcttttacctgcttgtagataacctctaatttatataatggttcaattatacagtggtaaatgtatctgggcttgtt
cttttaaaatagcaagtgtaacatgtgatagatgtagggtacttacaaagtactcaccaagcattgatgactgatgtcattagtatcatggct
aacattcactgagccctgattatgaggctggcacccaactaaatgttttgcattgtcacttcatcttcacaaagactttttgaataggttattct
ttatctccattttcagagaagcaactaagccctaaagaggtttattaacttgccaaataccacacagctagtaacactggtggggtctgga
atggaacccaggcaagctgaaaacgacagaacaatgaacactcctacttttcaattcgatttgctaattgttaatatttgccacactggctt
atctctctgtatccctacccatctctctctctctctgtctatatctatatgcaaacctggcatctaacttctaaatgcttgagcacggatctccta
agaacaagtacatggttctatgcaatgaaaagacatgattatggcatccaagagtgattttaaaaatcaaggatgaaacttaatttttaccc
acaagtccagacgatgatttctaccagtattctccagctttactctgtttcttatacttgacagactgagctccaagcctggaatcttctaaaa
agggtccactgccaatctggaatgccaacaaatcttattaatatatctccacatttgtaagcttctactaaattctccaagaacctgcatatc
agttaagtcctgaaatgctccagtgccaaataaaagcccactgtgcagatagaaatgcttttggtgactctcaaacatttattcaaaggtat
ttagtgagtatctgtcagctgtatatccagcactgagggaagactcagtctctgccatcacatacacctcagcactaagaagagacacat
gaaataattaccatccagtgtgataagtgctctgacagaggatggagtgaggactagtcaccttggggaagacagagaagcagggtc
ccaccaggagatgagaagccagcctccaggagtgcgctatctgagagggcaagagaaggaaggcaaagaggacgggtccatact
tgaacaacttgaagtaggtgtatctagtgcctaagagtgtgtatggtgacatcttgtggagcagtgaacatgaccctcatttggtttgttaa
agcattgggtcaaatgccacgggaatgtaacaggtaaatggtctttgccttataacacagggtatcccatccgtgtaagacaatgaatttt
taaaaaggcaaaaccaaaccttttgtttacatcatttgccttaaatagttcttagtacacaaattcttccttgcttggggccattatggtgtgtg
gaagtagaaaaagataaactgtagcttaaaacaaacaggtaacacataataagttccaaatgaggcattttgttgaaattcagtgttgagc
ataaacagtgaactcaaatgcacagaggaggcgtcatggaagggtgacaaaacactccagagcaataacagctaccctattttctgttc
cctgggagtagggagaaaagacatcaaaatcaaattataaatttaaaaaagtattaaaattctcttatggaaatatattaagaagtttgttag
gcaaatggaaaacgttttgtggaaacaagaaataagaaagaatggccatcacttaaagcaacagaatgtatctctcccactagctctcc
cctccctacccacagctttattcacatttgttctataacatttcagcacagttcagaccttccgctgagccagtgtgaggtcagggtgttga
ggaagcgagtccctgcagtagactccagctggctgtcgtcctgcttctgcttattccacagaatgagtctggtgggggagaggaaatt
cctaacctgtttttttttacacatcttcatcagaaaagcgaaagcactaattactgtccataataaaactactgggaaggttacttagcttgta
agtaagttccacaaagccatttcagtaaaaacttcctggttctaaaattgggaaaagcaggataatcctctaaggctgacattctttctctttt
gtgcatttacaactaaacggtcactgatacacatttccacagtgaccctgcaatatccacattggctttcttctaagacacttcaaaaataca
tcacacctttatgtgttaaatatcactattggcatatatctttaaaaacatttattttcttaattctcaaaatacagaaaagaatgttatgctattgt
gtgcacaaactgctattttcaataaggagctcttaatttagaaagttgctatttttcaataaagagctctcaattagaaaatatcaagaagtga
tgttaagtttttatggaaataaataaagtatcatgacatgaaaagtttataatttttcccccagcaatggcttactcagggaatttttcttatatgt
aaagttaatgatctgaaaagtggtaatcttatcattcaccaagatttcaggatataagttattcctacagaacaatccatttttacctttaaaga
tgagtttgtgtacttcagaagatgtgacgccagttggccaccatctcaatcagtagtaagtcacaccatcaatcccttgttaatgatggaac
tcatgtgaagtcaagaggaaacataatactctttacctcattccctggtagcagagaagttgactcatctttaatgtcagttattgagatgta
acacagaatcatattctacagaaaatatgtgataaactttacacaaatgaatacactttagaaaggcagtcatttggattttctttttgtcaga
gaacattgcaagtcttcctgaagaatttctgagaaattaaacactttatatgacattttacagcatgtagagaggttgaaactcagagaact
aaaactcagatgatagtctaaatacttaatttgttgctgctttatgctggacatgaaaagtgtgagcttaaattataaataaaaaactattgag
ctatcaaactatttggggtacatccttatctttcctctaaattactctttattcccattaaaatccacctatcccgggtcctctctcctcagccctt
cacctctgctttcaaagatgactaactaacctaataaacacatctaaggtgatgtgtcaacttcaaccccaaccctttttccccagATA
ACTTGTCTGCATTTCAAGAACAACCTACCAGAGACCTTACCTGTCACCTTGGCTC
TCCCACCCAATGGAGATGGCTCTAATGgtaaggaattctatcttaagtaaaagggaagggaaacacgctgac
agatgcagtttttataaaggcaggtcattggggcaccaagttggcagccgctttcaacttttgattctacctcaaaaggtaaccaagaaat
cgttgttattctaaagattatgacaagtccaaagtttcacagaatcctgatttttatacacttgcattaatcactgcttacttccatgatgtaaac
caaaactgcagcggcagaatatagagaatcagaataaattctgatagaaaaggcagatactgtttccacatatacaagtatatgctaatt
gcttttagcaaactgatccaaataggaatcaacattattccatcttaatgattatgtttttgattggtacacataaatgttaaagtgggaaaata
tgcagagaaagttacctaaactaaatcactttaattttcaacaacataattacattccatttcacctactaaaatggctttggacctagaatttg
tattgctacatcaatataagatgactttcagtaaaaatagatgtacagttgatttgtaacaacaaattaatagttttaaattatttgtatatttcaa
ggtaaataaaattagagctatcccacaatcgctttggttatgaaattcgaccactgtaactttttttaaagaaaactttatggtgtgcagttcct
ttggtatgcaaatattaaatggttggtgctgagaataagcagcaatatagaaatatatataattgtagaagaatataacctatgatttagaca
taaaatgagatccttacaagataaagagttatggagatggatgataatgatagttgtagaacattatgactaagtgtttaatatagcggacc
tgtacacttaaaaatggtgaagatgttaaacttacattatgtttacattacatatttcaccacaataaaaaatttttgaaggaaaaccagaact
ttaatgacaaacttaggacatattatgctctgaatatatagttttgataacgtctttcctggcaaatcaacaaggtaagaataaaaggatattc
tgaaatctttttttttctttggagacagagtcttgctctgttgcccaggctggagtgcagtggcacaatctcggctcactactaccttctgcct
cccaggttcaagtgatcctcctgcctcagcctcctgagtagctgggattataggcgtgcgccaccacgcctagctaatttttttgtattttta
gtagagacggggttttaccatgttggtcacgctggtctcaaactcctgacctcatgatccgcccacctcggcctcccaaaatgctaggat
tacaggcatgagacaccacacccagccagatattcttaaatcttatgctcacactccaccccccccaaaaaaattgtccttcaggaaagc
ataatttaaggaaaaatttcccaaagtgccaccttcagcttcattcttagcaccagaggaagaattgcctgtcatataatgaaagatccttg
gtgttatcattaatctagtatacatagtgcttcctcttggtagatttttacctactgtgagaaggaatgattttgatactcagacagtgtgtagta
atttgttaagaacatttaacttggaacctatgctacacagtaaaacttatactgacaatggatatttcatgtggtaggcctaagaagtattgtg
gagtgaggggactggaagtatatctgcagaactacctagtcaaagaccccagtctaatttctaatatataattcatataaattactcttgcat
tgctatacattaaacattatgcttgtcacattcttacttggttagcactgtcactactgattgtcaggcctataattgtgtataattcttcccctgc
cactttaccagctggtggccacaccgattccagaaagaactactttatttcttattacctcatgaaagcaccctctgcccagtgccttggct
aatagactggcctttgttgtctatgaccactaattcccactcaggcatatagcaattctgttgttggttgcttagcccagtcaaaaacactgg
aagtggacatagactccacactgatgaatgaatgaccttctagtcattctaatgtccttaaaagtctactatttttaaattgtagaatatatata
tatatatatatatttttttttttttctgggatatttctcctttcttctgttcattaatgtttttcatctatcattgtgttttaaatatgacctaaggttctatag
tatatttagacaaacttctcatttctctgcagtgttcggaattcatcatgcctgtatgacaaggttgtgtttgagaacaaaaggggacctgtgt
gacatgttttatttcaatatacatttagagtttgaacaaataaaaaaagctattagaatttttaatatatataacacattatcaaaaacactgtca
cttttctgagtgctctaatcaggcaattcgtatgtatatttaaaaagagggaaaaagctaattagtatactaaatatatttattttaatacctgtg
ctcatatgttatctttaaagaacaggaataaaatttatgggtagaatagtatgccagcaatttaccttcaatacaaattaaacacatcttttaa
gaaaagggcgagttatattggctggggtggcactaacactacattattaatatgtatttattgagcatttactgtgtccaggaccctgtaag
ggggaaaaaaagtccttgtttttacaatctttattgactgaaattctgtaaaatgaattgaattcctgaagtgacatcatttaacccaaatttttt
catcatcaactaattcatctcagaaaacagtaaagtttaaaaaaaaatactatcagtaagaataatatatttccttggtttggaaatttccaaa
ccacgatattctgccctgtcaaaatatattcactggggaattttttacttcatacgtttcttttgaacttagtcacatttttatatctagctggtttc
attattaaataaaagaaaaggtgattcccctcttgtactgcaggagaccacatacttgaatgatactctaacttctaggttctgttatagtaac
actgaaatcagaaacctgtgggagaaatgtcactttatctgagagagtaaaatcagactctaaaaggaagtagcaaattcatcttgttcttt
tttttaatcaaccctctgcctaagacaagtagtttgaaacacagaggactctttaagtcatacttcctttatgtagtcaaaaccaaagccact
gggtagtgctttccccaaaggaatctctaaatagtagacggggacattttcagatagattcgtttgtaggcaaacctccattgcttgtatca
catttcctgaaagaataaaggtaaaacttcaactatgtattacagaaagaaaaattcagcctgaaccctacccttataaaacaggttaattg
ggttttaattttcataaatcataaaggactattttgaacatttgggcctttaattgtctagctcctagatgaagtacaaatcagaaaaaaaaaa
aactgtactgtgtcagaatgcaagctttcctctttgcattttggcatttgaaaactccgaagagcggtttttgttttttatttaaagaagatgata
catatgtgtacccgattcaaaactagagaatagaatttaaaacataattttcaaagtcttcaaatatgcctaaaggtaacaatgtcatctttta
attgccaatttctctaccactttcaaaaaattacttccaaggatttaatgagctccttcctttcaacagaaaatggactattttcctttcagattta
ctatatgtgtcactccagctttataaccgcatgtgcatacacaaacatttcttttctcttgcagGTGGCACAAACCAGGA
AGGGGAAATCTGTGGTTTAAATTCTTTATGCCTCATCCTCTGAGTGCTGAAGGCT
TGCTGTAGGCTGTATGCTGTTAATGCTAATCGTGATAGGGGTTTTTGCCTCCAACT
GACTCCTACATATTAGCATTAACAGTGTATGATGCCTGTTACTAGCATTCACATG
GAACAAATTGCTGCCGTGGGAGGATGACAAAGAAGCATGAGTCACCCTGCTGGA
TAAACTTAGACTTCAGGCTTTATCATTTTTCAATCTGTTAATCATAATCTGGTCAC
TGGGATGTTCAACCTTAAACTAAGTTTTGAAAGTAAGGTTATTTAAAAGATTTAT
CAGTAGTATCCTAAATGCAAACATTTTCATTTAAATGTCAAGCCCATGTTTGTTTT
TATCATTAACAGAAAATATATTCATGTCATTCTTAATTGCAGGTTTTGGCTTGTTC
ATTATAATGTTCATAAACACCTTTGATTCAACTGTTAGAAATGTGGGCTAAACAC
AAATTTCTATAATATTTTTGTAGTTAAAAATTAGAAGGACTACTAACCTCCAGTT
ATATCATGGATTGTCTGGCAACGTTTTTTAAAAGATTTAGAAACTGGTACTTTCCC
CCAGGTAACGATTTTCTGTTCAGGCAACTTCAGTTTAAAATTAATACTTTTATTTG
ACTCTTAAAGGGAAACTGAAAGGCTATGAAGCTGAATTTTTTTAATGAAATATTT
TTAACAGTTAGCAGGGTAAATAACATCTGACAGCTAATGAGATATTTTTTCCATA
CAAGATAAAAAGATTTAATCAAAAAATTTCATATTTGAAATGAAGTCCCAAATCT
AGGTTCAAGTTCAATAGCTTAGCCACATAATACGGTTGTGCGAGCAGAGAATCT
ACCTTTCCACTTCTAAGCCTGTTTCTTCCTCCATATGGGGATAATACTTTACAAGG
TTGTTGTGAGGCTTAGATGAGATAGAGAATTATTCCATAAGATAATCAAGTGCTA
CATTAATGTTATAGTTAGATTAATCCAAGAACTAGTCACCCTACTTTATTAGAGA
AGAGAAAAGCTAATGATTTGATTTGCAGAATATTTAAGGTTTGGATTTCTATGCA
GTTTTTCTAAATAACCATCACTTACAAATATGTAACCAAACGTAATTGTTAGTAT
ATTTAATGTAAACTTGTTTTAACAACTCTTCTCAACATTTTGTCCAGGTTATTCAC
TGTAACCAAATAAATCTCATGAGTCTTTAGTTGATTTAAAATAA

1. A genetically modified cell comprising a deletion in a miR-155 host gene or a fragment thereof.

2. The genetically modified cell of claim 1, wherein the deletion is in exon 1, exon 2, exon 3, or the transcriptional start site of the miR-155 host gene.

3. The genetically modified cell of claim 1, wherein the deletion is in exon 3.

4. The genetically modified cell of claim 1, wherein the genetically modified cell is engineered using a method comprising introducing into the cell a CRISPR/Cas endonuclease (Cas)9 system with one or more CRISPR/Cas guide RNAs, wherein the one or more guide RNAs target the miR-155 host gene or a fragment thereof.

5. The genetically modified cell of claim 4, wherein the one or more guide RNAs target exon 1, exon 2, exon 3, intron 1, intron 2, or the transcriptional start site of the miR-155 host gene.

6. The genetically modified cell of claim 4, wherein the guide RNA comprises a polynucleotide sequence at least 80% identical to one of SEQ ID NOs: 1-14.

7. The genetically modified cell of claim 4, wherein the CRISPR/Cas9 system comprises a first guide RNA and a second guide RNA.

8. The genetically modified cell of claim 7, wherein the first guide RNA comprises a polynucleotide sequence at least 80% identical to SEQ ID NO: 11 or 13 and the second guide RNA comprises a polynucleotide sequence at least 80% identical to SEQ ID NO: 12 or 14.

9. The genetically modified cell of claim 4, wherein the target sequence of the one or more CRISPR/Cas guide RNAs comprises a polynucleotide sequence at least 80% identical to SEQ ID NOs: 35-39.

10. The genetically modified cell of claim 1, wherein the genetically modified cell is a T cell or a stem cell.

11. The genetically modified cell of claim 10, wherein the T cell is a primary T cell, a T cell line, a chimeric antigen receptor (CAR)-T cell, a tumor infiltrating lymphocyte, an effector T cell, a memory T cell, a TEMRA, or a stem cell-like memory T cell.

12. The genetically modified cell of claim 10, wherein the T cell is a tumor-specific T cell.

13. The genetically modified cell of claim 10, wherein the T cell is an activated T cell.

14. A method for preventing or treating acute graft-versus-host disease in a subject, comprising administering to the subject a therapeutically effective amount of the genetically modified cell of claim 1.

15. The method of claim 14, wherein the genetically modified cell is a T cell.

16. The method of claim 15, wherein the T cell is a primary T cell, a T cell line, a chimeric antigen receptor (CAR)-T cell, a tumor infiltrating lymphocyte, an effector T cell, a memory T cell, a TEMRA, or a stem cell-like memory T cell.

17. The method of claim 15, wherein the T cell is a tumor-specific T cell.

18. The method of claim 15, wherein the T cell is an activated T cell.

19. (canceled)

20. (canceled)

21. (canceled)

22. (canceled)

23. (canceled)

24. A method of creating a genetically modified T cell comprising a deletion in a miR-155 host gene, said method comprising:
obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.

25-35. (canceled)

36. A method for preventing or treating acute graft-versus-host disease in a subject, comprising administering to the subject a genetically modified T cell comprising a deletion in a miR-155 host gene, wherein the genetically modified T cell is created by a method comprising:
obtaining a T cell; and
introducing into the T cell a CRISPR/Cas endonuclease (Cas)9 system with one or more CRISPR/Cas guide RNAs, wherein the one or more guide RNAs target the miR-155 host gene or a fragment thereof.


SEQUENCE LISTING (45 sequences)

SEQ ID NO: 1 - RNA, 20 nt, Artificial Sequence
  Synthetic Construct
guuaaugcuaaucgugauag

SEQ ID NO: 2 - RNA, 20 nt, Artificial Sequence
  Synthetic Construct
gcuaauauguaggagucagu

SEQ ID NO: 3 - RNA, 20 nt, Artificial Sequence
  Synthetic Construct
accgaagucccauaugcucc

SEQ ID NO: 4 - RNA, 20 nt, Artificial Sequence
  Synthetic Construct
cacguuuuacgagggagagc

SEQ ID NO: 5 - RNA, 20 nt, Artificial Sequence
  Synthetic Construct
ucuugcagguggcacaaacc

SEQ ID NO: 6 - RNA, 20 nt, Artificial Sequence
  Synthetic Construct
gccuguuacuagcauucaca

SEQ ID NO: 7 - RNA, 20 nt, Artificial Sequence
  Synthetic Construct
gacaucccgaguauaaaugc

SEQ ID NO: 8 - RNA, 20 nt, Artificial Sequence
  Synthetic Construct
gucuccagcugauucggucc

SEQ ID NO: 9 - RNA, 20 nt, Artificial Sequence
  Synthetic Construct
acgucgcccgaacuuucagu

SEQ ID NO: 10 - RNA, 20 nt, Artificial Sequence
  Synthetic Construct
uaaaagggucgcacguucgc

SEQ ID NO: 11 - RNA, 23 nt, Artificial Sequence
  Synthetic Construct
gacaucccgaguauaaaugcggg

SEQ ID NO: 12 - RNA, 23 nt, Artificial Sequence
  Synthetic Construct
acgucgcccgaacuuucagucgg

SEQ ID NO: 13 - RNA, 23 nt, Artificial Sequence
  Synthetic Construct
ucuugcagguggcacaaaccagg

SEQ ID NO: 14 - RNA, 23 nt, Artificial Sequence
  Synthetic Construct
auggaacaaauugcugccguggg

SEQ ID NO: 15 - DNA, 20 bp, Artificial Sequence
  Synthetic Construct
aaatcaaatggctgctccag

SEQ ID NO: 16 - DNA, 20 bp, Artificial Sequence
  Synthetic Construct
gggctccaacctttgttctt

SEQ ID NO: 17 - DNA, 20 bp, Artificial Sequence
  Synthetic Construct
tgagctccttcctttcaaca

SEQ ID NO: 18 - DNA, 20 bp, Artificial Sequence
  Synthetic Construct
tgaacatcccagtgaccaga

SEQ ID NO: 19 - DNA, 20 bp, Artificial Sequence
  Synthetic Construct
atctaggggtgaacgacacg

SEQ ID NO: 20 - DNA, 20 bp, Artificial Sequence
  Synthetic Construct
gaatacccatcttgccctga

SEQ ID NO: 21 - DNA, 20 bp, Artificial Sequence
  Synthetic Construct
ctcagggcacttctgtagcc

SEQ ID NO: 22 - DNA, 20 bp, Artificial Sequence
  Synthetic Construct
tggaaggaaccccataacaa

SEQ ID NO: 23 - DNA, 20 bp, Artificial Sequence
  Synthetic Construct
gtgtgtggacaggtgtgagg

SEQ ID NO: 24 - DNA, 20 bp, Artificial Sequence
  Synthetic Construct
gcctcggagacactgaaaag

SEQ ID NO: 25 - DNA, 20 bp, Artificial Sequence
  Synthetic Construct
taggggtggcttaggttctg

SEQ ID NO: 26 - DNA, 16 bp, Artificial Sequence
  Synthetic Construct
aggaacccccaagtcc

SEQ ID NO: 27 - DNA, 20 bp, Artificial Sequence
  Synthetic Construct
caggattgcctgagctttgt

SEQ ID NO: 28 - DNA, 21 bp, Artificial Sequence
  Synthetic Construct
gctgttgaggacaagaaatgc

SEQ ID NO: 29 - DNA, 20 bp, Artificial Sequence
  Synthetic Construct
gagcccagctctgaaaagtg

SEQ ID NO: 30 - DNA, 20 bp, Artificial Sequence
  Synthetic Construct
tcagcctagggaagagacca

SEQ ID NO: 31 - DNA, 20 bp, Artificial Sequence
  Synthetic Construct
tgggctcctgactctcagtt

SEQ ID NO: 32 - DNA, 20 bp, Artificial Sequence
  Synthetic Construct
gctggagacttctggacctg

SEQ ID NO: 33 - DNA, 20 bp, Artificial Sequence
  Synthetic Construct
gctttttcccctttcctgtc

SEQ ID NO: 34 - DNA, 20 bp, Artificial Sequence
  Synthetic Construct
catattggccaggatggtct

SEQ ID NO: 35 - DNA, 23 bp, Artificial Sequence
  Synthetic Construct
gacatcccgagtataaatgcggg

SEQ ID NO: 36 - DNA, 23 bp, Artificial Sequence
  Synthetic Construct
gtctccagctgattcggtccagg

SEQ ID NO: 37 - DNA, 23 bp, Artificial Sequence
  Synthetic Construct
ccgactgaaagttcgggcgacgt

SEQ ID NO: 38 - DNA, 23 bp, Artificial Sequence
  Synthetic Construct
tcttgcaggtggcacaaaccagg

SEQ ID NO: 39 - DNA, 23 bp, Artificial Sequence
  Synthetic Construct
atggaacaaattgctgccgtggg

SEQ ID NO: 40 - DNA, 106 bp, Artificial Sequence
  Synthetic Construct
tacacacacgcaatgacccacgagaaagggaaaggggaaaacaccaactacccgggcgctgggctttttcgacttttcctttaaaaagaaaaaagtttttcaagct

SEQ ID NO: 41 - DNA, 133 bp, Homo sapiens
gcgcgggcttcctgtgcgcggccgagcccgggcccagcgccgcctgcagcctcgggaagggagcggatagcggagccccgagccgcccgcagagcaagcgcggggaaccaaggagacgctcctggcactgcag

SEQ ID NO: 42 - DNA, 85 bp, Homo sapiens
ataacttgtctgcatttcaagaacaacctaccagagaccttacctgtcaccttggctctcccacccaatggagatggctctaatg

SEQ ID NO: 43 - DNA, 1276 bp, Homo sapiens
gtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaaatttcatatttgaaatgaagtcccaaatctaggttcaagttcaatagcttagccacataatacggttgtgcgagcagagaatctacctttccacttctaagcctgtttcttcctccatatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgatttaaaataa

SEQ ID NO: 44 - DNA, 62 bp, Homo sapiens
ttaatgctaatcgtgataggggtttttgcctccaactgactcctacatattagcattaacag

SEQ ID NO: 45 - DNA, 16210 bp, Homo sapiens
gatcaagatgtcagcaggactggttcctcctgagagctgtgagggaaggatctgtttcaagcccctctccttggctggtagatggccatcttttcccaatgtttcttcacactgtcttccttctatatgtgtttctttctctacacatcatgttctttttctaagaacaccagtcatattggattaggagccaaccctattttaataagacctcatcttgactaattacatccacaaaggccttgtttccacataaggttatattctgaagtactggttggtctacagccataccaccccacacacacccgatctcatctgaagtactgggtgttaggacatcaatatatgaatttgggaggtggggggagggggacacaattcagcccacaacaacatccatggggtacttgtcccggcaggtggccagctcttactttctcaagctatccatttggagcaggtccttttttttttttctttttttttttttttgagacagtctcgctcgcattccaggctggaatgcagtggcgcgatcttggctcaccgcaaactccgcctcccgcattcatgccattctcctgcctcagcctccagagtagctgggactacaggtgcccgccatcatgcccggctaatttttttttcttttttgtatttttagtagattcagggtttcaccattctagccaggatggtctcaatctcctgacctcatgatccagtcacctcagcctcccaaagtgctgggattacaggatgagccaccgcgcctggcctggagcaggtactttttatccatcaccttatttaatcatcatatccctcttagtctgctagggttgccataagaaaataccatagactggatggctgatacaacaacaatttattttctcacattctggaggctagaaagttccagatcaaggtcctgcagggttcggtttctggtgaggactctcttcctggcttacaggaaaccaccatcccattgtgtgctcacatgacctctgctttgactgatttctgagagaaagggaattctggtgtctcttcctcttctcataagagcactagctctatgggattagtgatccacccttttgacctcatttaacctttatcacctcctcacaggccctatctccaaatggggataacattggggatcagggcttcaacatataaagttgaagagaacatgattcagtctatagtaatccctgatgggagacatcattattgtcatttacaggaagctgagacataaaaagtttaacgttcagattccacagctagaattagtgtaaaatgcaggtgtatttgactcctaaaacccatttatccatccactattctgggtgtctcaaactgggacctaaaatgcggaggtttgcacatgacctgaaattacttccaccactgggagaagggaggattgagcaatttcctgtttctattaaagtgcattatacaaacttgtcattctggggatgaaaggtcaccctagaattgcctatgggcaatttcttatagttcaacctagaatgagaaatgggaaattcagaaaggcattgtaggcatctgtaaccagcagagggacgtgccccacctgggtggggaccatgcatccttgccacatgccccactgcacaacttccccagctcctcaacgtcacatggatctggaaagcagggagactggactacggagccagccctccagggttggaactgagtttgaatctcagcttgactacttactaggatctaaggtaacatgtttaatctctctgtgctcagtttcctcatctggaaatagagattataatgctcctccctcatatgatcatagtgaggactgaaagagttaatgcatataaggagtgttaggacaggatctggcacatggtaaatgctttataagtgttagttgttatcatcatgattattcttgtttgaattataagagaaaaagcatgtatcttaagaatagagggttttaaaatgctcccaagttccttaaccaacctgagccatctgtaaattaagtactatgggatttccagctctgacatgtattctgacatgtaactggcatcagtttaatacaaactattctaaaatgtcttgtgcccttgacagggaggtccaagtactggatacttgcaatgcaatccagcagtggccccgtctttcttacaaaaaggccccagtcacatgttgatgaggctagatctattcctgtcctccttctcttccccatatttccttattcttcttcaagtctaaacgtttattgaactgagacccatgaatgagttactcgactaggcttgtaggataaacttgccaggctttagatctatttgttcctttccatcccccaaaatcaaatggctgctccaggaaaatgttccccttgtggcagggtccgggagaaaagagagaagcgacaaaaccaaaaattaaaacgaccgaagtcccatatgctccaggaatatgtcctggagatgggagtggagggcagggggagaatgttgttgaggtcaaaatttttgaagttttaagtcctatatcttgacatcccgagtataaatgcgggtaccagacacagtacaaacgttctcaaagcccagttacgtattccaaaccaaacgcgggctcttgaagggtgatgaggtagggatgaaatccaggatcgcctgaagaccatttcttcctctcttagggacctgctggtctccagctgattcggtccaggaggaaaaacctcccacttgctcctctcgggctccctgcaaggagagagtagagacactcctgccacccagttgcaagaagtcgccacttccccctccagccgactgaaagttcgggcgacgtctgggccgtcatttgaaggcgtttccttttctttaagaacaaaggttggagcccaagccttgcggcgcggtgcaggaaagtacacggcgtgtgttgagagaaaaaaaatacacacacgcaatgacccacgagaaagggaaaggggaaaacaccaactacccgggcgctgggctttttcgacttttcctttaaaaagaaaaaagtttttcaagctgtaggttccaagaacaggcaggaggggggagaagggggggggggttgcagaaaaggcgcctggtcggttatgagtcacaagtgagttataaaagggtcgcacgttcgcaggcgcgggcttcctgtgcgcggccgagcccgggcccagcgccgcctgcagcctcgggaagggagcggatagcggagccccgagccgcccgcagagcaagcgcggggaaccaaggagacgctcctggcactgcaggtacgccgacttcagtctcgcgctcccgcccgcctttcctctcttgaacgtggcagggacgccgggggacttcggtgcgagggtcaccgccgggttaactggcgaggcaaggcgggggcagcgcgcacgtggccgtggagcccggcctggtcccgcgcgcgcctgcgggtgccccctggggactcagtggtgtcgcctcgcccgggaccagagattgcgctggatggattcccgcgggcagaggcagggggaaggaggggtgttcgaaacctaatacttgagcttctttgcaaagtttccttggatggttggggacgtacctgtataatggccctggaccagcttccctgttggagtggccagagaagtgtgtaaaacacactagaggggcagggtggaaaaagagactgccttcaaaacttgtatcttttcgatttcattttgaaaaataactacaaatctattttaattttacaaagttagactcatagcattttagatatcaatgtcttcatttaacagaagtgaagatggagcaaacgctcaatcagcgtctgtatttattcgctcctgttgtgccagggtgcgtttttgccgagcggttgcctttctttactcacaaaacccccttgatgtctgtcctccacgttttacgagggagagccggatcttttgaagtttgtatcatctaaagcaggtatattgggatgactatggatagaatttaacctgaaaacactgaagttgacagctgacaaagatataagaatccaaagtatgttaaaaattaaggagctgaggcccacagaagtgaagttacttttccagcatcacacagcagatctaggaccctgtgtgatttgaaaaggccaaggtatctaatgatgctcataatttagtttacattttgtgtcaaatagatgaggtcaagtggacattatgtgtatattccaaagccaaaattactgcttgtaatatccattcatctctccgaagacgtagtgcctcttttatatggctttaacctaatgtataatcgaagtgaattagggcagccagacttttaatcacaggactgatcttttaaaaacgggtatatgtaaagttaattgcttatctatgtgaacagtaatatacactataaaaagttttcaaggcaacttcctgcctgtggtttaatttgatagtcacaatgataggaaattggcagggttaggtggtggtatctacattttgcccatgaggacttgtggctcagagaatttgagttttttctagtaaggtcactaggtccctagtgcacagcctgcttttaggtacggatgtgggatgggggtgtcggcagaccacagggttttgactctcagtttgggctcatttccatgactccttgctgcctccctgatttctgtcaaatgaccaaagcacatttagtaattacttctgctgcaaggactactccttattaggctgtgataagtaagtttcctcacatagtgggtcagctcactctggccacaggacccagcttcctaaccacacacattaagaaagagaaaaattagcactgtctgagacctacaaccacttcaagggagaacagtggtgtttgcacaaatgtctacttttgttttaacctagtcatgtgtaaaaagtgtaattcactggtctttaccaaaaaaaaaaaaaaaaaaacaacaacaaaaaaaaaacaggcatcatagtctgcctgtgctggacgctatgctagacactggagagacaaaggtgagcaggataggcacaaccctacccttacaaacccagaaagttagaggaaaggccatcaggacattgctgtgcaatttgatatcatttcaattttggtaacatagatatttgaactaattagaatgtgctacaaattttgccattctttactttccatatgccaacatggaaaatttgtaaccctcatctttaaatacctcatcttaccagtcctctataaatcaaaaaatctaccccaaaaagccctaggttttaacttttgaaaagtttttttttatcagcttttcaggtcatgactaagatctgccattcaatttaaaggaattctttcattttagagtaatagttactaaaatcctttattcctacctccttgagaatctaatggaaattttatatgctcttcccagaaaatttttgttaatgtgtatcctcacacaccaatttgcattcgatgttagcaggctagagacttccttgaatctcagccatgaccctcttcgggggtccactttcataatcagtattctaaacctagtaggcataaatagcttgaatagaggaaagtggtatgtagactcttgcccttcacttggactgggcacctctatcacttggactgggcaccagctagtgcccttaactagctggtgtctccatttttgatagtcattggctattgcttagctgaagctattgattttaacagccttattgttttgatatcaggctgccactctgcagaagtctaatactggggaaggagcaccggcctgagaaccatggtttgtttctaggaccaactctgcagatttctgtgcctcattttgctcatctctaagatgggtggttgatgggtggcagggactgaaccatttctaaaatcccttacactgatgaaattctgtgattccagtgtaactaccaagctcactttcatatacctcctgagacacagctggtcttagtgaatttatatcctctatgtccaaagtaagtcaaagtaagtaccactataattcactatagaatgtatttttgtctttcactgatcttactggactcaaaactgttacatgtttccttaaattagatagaataaaaaaattgtgaaacactacttttcaaggaaaaatattacaattttaaaagcctctttttatcccacctctcagtagctacaactttcatatacgagtttcacatatatatgaaaaaaattttcttatctcagactcttaaggaaatgcaatgcttttaatatgatcactgctgtgtaattctcatctgccctgaaacacttgttctgtccatcaatcaatgtatttgggtttacaatatgcagagcctcaagcaaaaccagtgcaaaagcacatctatcctcaaataggcacatcatatgtctgagcagttcagcatcctcactcacaagtccaaggctctcagcaatgtggaagaagatgtgattcctaaaattaccacccttacacacagtgacacactcaccagaaagtgaggcttaacatagcttaagtggctgtagtttaaaacagattgtgaatatctgtgctaattctgagaaacaattagcagacatattaaccaattagcacagatcactaatatctgtgctaattgttaggacagattagcacagatattgctaccttgtgtgggtagacagggttaaggcactggcagaaggaaagattagtgaggaatatcaaatagctgatttaatgaaattgtgaggccgatgtttggataaggtgaaacagtgccctacaatgacatttatgccagttctccataaacaagagtgttctacacaaattataagttcctggctgtcttaacaaaatgctatggtcctttatatgctcacacaaataatggttccaagcatgggaaacaggtgccagattgcccaggtaggcatcccagcagcatgctccttagccactgattaaccttgcgcaagttatttcacctgcctgtgcctcaaggcccctgtgtttcaatggggagggtgtgaatactcattcctaaggttgtgaggaggaataactgagttagtatacgtcaaacacttaggtgcttggcacagagggagcctgtgtaagtgtttgctattattttgtttcactagatgatcttgtgtattataagaaatcaatttggaaggtgagatacaaatgcagtctgagaaagtaaagacgttttaaagacattttactgtgtcatgaattttcagagaccacactgacatggtaaaaagttgtttggtttttagaatttttgtttcagaatgcatatccttctccttaaccaaaagaaaaactactaaaatacttctgaaaattatttccaagaaaacattttggtgtaccttttagcagttactttatgacttaaagattaggatgaacaaaataattattttaacttggcttttacctgcttgtagataacctctaatttatataatggttcaattatacagtggtaaatgtatctgggcttgttcttttaaaatagcaagtgtaacatgtgatagatgtagggtacttacaaagtactcaccaagcattgatgactgatgtcattagtatcatggctaacattcactgagccctgattatgaggctggcacccaactaaatgttttgcattgtcacttcatcttcacaaagactttttgaataggttattctttatctccattttcagagaagcaactaagccctaaagaggtttattaacttgccaaataccacacagctagtaacactggtggggtctggaatggaacccaggcaagctgaaaacgacagaacaatgaacactcctacttttcaattcgatttgctaattgttaatatttgccacactggcttatctctctgtatccctacccatctctctctctctctgtctatatctatatgcaaacctggcatctaacttctaaatgcttgagcacggatctcctaagaacaagtacatggttctatgcaatgaaaagacatgattatggcatccaagagtgattttaaaaatcaaggatgaaacttaatttttacccacaagtccagacgatgatttctaccagtattctccagctttactctgtttcttatacttgacagactgagctccaagcctggaatcttctaaaaagggtccactgccaatctggaatgccaacaaatcttattaatatatctccacatttgtaagcttctactaaattctccaagaacctgcatatcagttaagtcctgaaatgctccagtgccaaataaaagcccactgtgcagatagaaatgcttttggtgactctcaaacatttattcaaaggtatttagtgagtatctgtcagctgtatatccagcactgagggaagactcagtctctgccatcacatacacctcagcactaagaagagacacatgaaataattaccatccagtgtgataagtgctctgacagaggatggagtgaggactagtcaccttggggaagacagagaagcagggtcccaccaggagatgagaagccagcctccaggagtgcgctatctgagagggcaagagaaggaaggcaaagaggacgggtccatacttgaacaacttgaagtaggtgtatctagtgcctaagagtgtgtatggtgacatcttgtggagcagtgaacatgaccctcatttggtttgttaaagcattgggtcaaatgccacgggaatgtaacaggtaaatggtctttgccttataacacagggtatcccatccgtgtaagacaatgaatttttaaaaaggcaaaaccaaaccttttgtttacatcatttgccttaaatagttcttagtacacaaattcttccttgcttggggccattatggtgtgtggaagtagaaaaagataaactgtagcttaaaacaaacaggtaacacataataagttccaaatgaggcattttgttgaaattcagtgttgagcataaacagtgaactcaaatgcacagaggaggcgtcatggaagggtgacaaaacactccagagcaataacagctaccctattttctgttccctgggagtagggagaaaagacatcaaaatcaaattataaatttaaaaaagtattaaaattctcttatggaaatatattaagaagtttgttaggcaaatggaaaacgttttgtggaaacaagaaataagaaagaatggccatcacttaaagcaacagaatgtatctctcccactagctctcccctccctacccacagctttattcacatttgttctataacatttcagcacagttcagaccttccgctgagccagtgtgaggtcagggtgttgaggaagcgagtccctgcagtcagactccagctggctgtcgtcctgcttctgcttattccacagaatgagtctggtgggggagaggaaattcctaacctgtttttttttacacatcttcatcagaaaagcgaaagcactaattactgtccataataaaactactgggaaggttacttagcttgtaagtaagttccacaaagccatttcagtaaaaacttcctggttctaaaattgggaaaagcaggataatcctctaaggctgacattctttctcttttgtgcatttacaactaaacggtcactgatacacatttccacagtgaccctgcaatatccacattggctttcttctaagacacttcaaaaatacatcacacctttatgtgttaaatatcactattggcatatatctttaaaaacatttattttcttaattctcaaaatacagaaaagaatgttatgctattgtgtgcacaaactgctattttcaataaggagctcttaatttagaaagttgctatttttcaataaagagctctcaattagaaaatatcaagaagtgatgttaagtttttatggaaataaataaagtatcatgacatgaaaagtttataatttttcccccagcaatggcttactcagggaatttttcttatatgtaaagttaatgatctgaaaagtggtaatcttatcattcaccaagatttcaggatataagttattcctacagaacaatccatttttacctttaaagatgagtttgtgtacttcagaagatgtgacgccagttggccaccatctcaatcagtagtaagtcacaccatcaatcccttgttaatgatggaactcatgtgaagtcaagaggaaacataatactctttacctcattccctggtagcagagaagttgactcatctttaatgtcagttattgagatgtaacacagaatcatattctacagaaaatatgtgataaactttacacaaatgaatacactttagaaaggcagtcatttggattttctttttgtcagagaacattgcaagtcttcctgaagaatttctgagaaattaaacactttatatgacattttacagcatgtagagaggttgaaactcagagaactaaaactcagatgatagtctaaatacttaatttgttgctgctttatgctggacatgaaaagtgtgagcttaaattataaataaaaaactattgagctatcaaactatttggggtacatccttatctttcctctaaattactctttattcccattaaaatccacctatcccgggtcctctctcctcagcccttcacctctgctttcaaagatgactaactaacctaataaacacatctaaggtgatgtgtcaacttcaaccccaaccctttttccccagataacttgtctgcatttcaagaacaacctaccagagaccttacctgtcaccttggctctcccacccaatggagatggctctaatggtaaggaattctatcttaagtaaaagggaagggaaacacgctgacagatgcagtttttataaaggcaggtcattggggcaccaagttggcagccgctttcaacttttgattctacctcaaaaggtaaccaagaaatcgttgttattctaaagattatgacaagtccaaagtttcacagaatcctgatttttatacacttgcattaatcactgcttacttccatgatgtaaaccaaaactgcagcggcagaatatagagaatcagaataaattctgatagaaaaggcagatactgtttccacatatacaagtatatgctaattgcttttagcaaactgatccaaataggaatcaacattattccatcttaatgattatgtttttgattggtacacataaatgttaaagtgggaaaatatgcagagaaagttacctaaactaaatcactttaattttcaacaacataattacattccatttcacctactaaaatggctttggacctagaatttgtattgctacatcaatataagatgactttcagtaaaaatagatgtacagttgatttgtaacaacaaattaatagttttaaattatttgtatatttcaaggtaaataaaattagagctatcccacaatcgctttggttatgaaattcgaccactgtaactttttttaaagaaaactttatggtgtgcagttcctttggtatgcaaatattaaatggttggtgctgagaataagcagcaatatagaaatatatataattgtagaagaatataacctatgatttagacataaaatgagatccttacaagataaagagttatggagatggatgataatgatagttgtagaacattatgactaagtgtttaatatagcggacctgtacacttaaaaatggtgaagatgttaaacttacattatgtttacattacatatttcaccacaataaaaaatttttgaaggaaaaccagaactttaatgacaaacttaggacatattatgctctgaatatatagttttgataacgtctttcctggcaaatcaacaaggtaagaataaaaggatattctgaaatctttttttttctttggagacagagtcttgctctgttgcccaggctggagtgcagtggcacaatctcggctcactactaccttctgcctcccaggttcaagtgatcctcctgcctcagcctcctgagtagctgggattataggcgtgcgccaccacgcctagctaatttttttgtatttttagtagagacggggttttaccatgttggtcacgctggtctcaaactcctgacctcatgatccgcccacctcggcctcccaaaatgctaggattacaggcatgagacaccacacccagccagatattcttaaatcttatgctcacactccaccccccccaaaaaaattgtccttcaggaaagcataatttaaggaaaaatttcccaaagtgccaccttcagcttcattcttagcaccagaggaagaattgcctgtcatataatgaaagatccttggtgttatcattaatctagtatacatagtgcttcctcttggtagatttttacctactgtgagaaggaatgattttgatactcagacagtgtgtagtaatttgttaagaacatttaacttggaacctatgctacacagtaaaacttatactgacaatggatatttcatgtggtaggcctaagaagtattgtggagtgaggggactggaagtatatctgcagaactacctagtcaaagaccccagtctaatttctaatatataattcatataaattactcttgcattgctatacattaaacattatgcttgtcacattcttacttggttagcactgtcactactgattgtcaggcctataattgtgtataattcttcccctgccactttaccagctggtggccacaccgattccagaaagaactactttatttcttattacctcatgaaagcaccctctgcccagtgccttggctaatagactggcctttgttgtctatgaccactaattcccactcaggcatatagcaattctgttgttggttgcttagcccagtcaaaaacactggaagtggacatagactccacactgatgaatgaatgaccttctagtcattctaatgtccttaaaagtctactatttttaaattgtagaatatatatatatatatatatatttttttttttttctgggatatttctcctttcttctgttcattaatgtttttcatctatcattgtgttttaaatatgacctaaggttctatagtatatttagacaaacttctcatttctctgcagtgttcggaattcatcatgcctgtatgacaaggttgtgtttgagaacaaaaggggacctgtgtgacatgttttatttcaatatacatttagagtttgaacaaataaaaaaagctattagaatttttaatatatataacacattatcaaaaacactgtcacttttctgagtgctctaatcaggcaattcgtatgtatatttaaaaagagggaaaaagctaattagtatactaaatatatttattttaatacctgtgctcatatgttatctttaaagaacaggaataaaatttatgggtagaatagtatgccagcaatttaccttcaatacaaattaaacacatcttttaagaaaagggcgagttatattggctggggtggcactaacactacattattaatatgtatttattgagcatttactgtgtccaggaccctgtaagggggaaaaaaagtccttgtttttacaatctttattgactgaaattctgtaaaatgaattgaattcctgaagtgacatcatttaacccaaattttttcatcatcaactaattcatctcagaaaacagtaaagtttaaaaaaaaatactatcagtaagaataatatatttccttggtttggaaatttccaaaccacgatattctgccctgtcaaaatatattcactggggaattttttacttcatacgtttcttttgaacttagtcacatttttatatctagctggtttcattattaaataaaagaaaaggtgattcccctcttgtactgcaggagaccacatacttgaatgatactctaacttctaggttctgttatagtaacactgaaatcagaaacctgtgggagaaatgtcactttatctgagagagtaaaatcagactctaaaaggaagtagcaaattcatcttgttcttttttttaatcaaccctctgcctaagacaagtagtttgaaacacagaggactctttaagtcatacttcctttatgtagtcaaaaccaaagccactgggtagtgctttccccaaaggaatctctaaatagtagacggggacattttcagatagattcgtttgtaggcaaacctccattgcttgtatcacatttcctgaaagaataaaggtaaaacttcaactatgtattacagaaagaaaaattcagcctgaaccctacccttataaaacaggttaattgggttttaattttcataaatcataaaggactattttgaacatttgggcctttaattgtctagctcctagatgaagtacaaatcagaaaaaaaaaaaactgtactgtgtcagaatgcaagctttcctctttgcattttggcatttgaaaactccgaagagcggtttttgttttttatttaaagaagatgatacatatgtgtacccgattcaaaactagagaatagaatttaaaacataattttcaaagtcttcaaatatgcctaaaggtaacaatgtcatcttttaattgccaatttctctaccactttcaaaaaattacttccaaggatttaatgagctccttcctttcaacagaaaatggactattttcctttcagatttactatatgctgtcactccagctttataaccgcatgtgcatacacaaacatttctttctctcttgcaggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaaatttcatatttgaaatgaagtcccaaatctaggttcaagttcaatagcttagccacataatacggttgtgcgagcagagaatctacctttccacttctaagcctgtttcttcctccatatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgatttaaaataa